(19) United States
(12) Patent Application Publication
(10) Pub. No.: US 2026/0078142 A1
(43) Pub. Date: Mar. 19, 2026

(54) FULLY-CONTINUOUS SYNTHESIS METHOD OF CYPROTERONE ACETATE

(71) Applicant: Fudan University, Shanghai (CN)

(72) Inventors: Fener CHEN, Shanghai (CN); Yajiao ZHANG, Shanghai (CN); Li WAN, Shanghai (CN); Minjie LIU, Shanghai (CN); Xianjing ZHENG, Shanghai (CN); Liang GAO, Shanghai (CN); Dang CHENG, Shanghai (CN)

(21) Appl. No.: 19/402,490

(22) Filed: Nov. 26, 2025

(30) Foreign Application Priority Data
Nov. 29, 2024 (CN) 202411742232.4

(51) Int. Cl. C07J 53/00; B01J 31/00; B01J 31/02; B01J 31/22

(52) U.S. Cl. CPC C07J 53/004; B01J 31/003; B01J 31/0225; B01J 31/2234; B01J 2531/845

(57) ABSTRACT

A fully-continuous synthesis method of cyproterone acetate is provided. 4-androstene-3,17-dione is adopted as a starting material. The present disclosure adopts a fully continuous device composed of micromixers, microreactors, online gravity separation units, online solvent switching units, online solvent concentration units and solvent recovery systems connected according to a cyproterone acetate synthesis route. Cyproterone acetate product is synthesized through one enzymatic catalytic reaction, nine chemical reactions and continuous operations. This can realize recovery of dichloromethane, dichloroethane and ethanol, and significantly reduce emission of three wastes. The obtained crude cyproterone acetate is subjected to decolorization, recrystallization, filtration and drying to obtain pure cyproterone acetate with a purity greater than 99%.

### CROSS-REFERENCE TO RELATED APPLICATIONS

This application claims the benefit of priority from Chinese Patent Application No. 202411742232.4, filed on Nov. 29, 2024. The content of the aforementioned application, including any intervening amendments made thereto, is incorporated herein by reference in its entirety.

### TECHNICAL FIELD

This application relates to chemical and pharmaceutical technology, and more particularly to a fully-continuous synthesis method of cyproterone acetate.

### BACKGROUND

Cyproterone acetate, commonly known as “Cyproterone”, can potently antagonize androgen receptors and reduce serum testosterone due to its weak anti-gonadotropic effect. It was first used clinically as an anti-androgen in 1964. Regarded as the only hormone capable of inducing complete androgen blockade through monotherapy, cyproterone acetate is clinically used for the treatment of advanced prostate cancer, metastatic male breast cancer, and symptoms such as acne and alopecia caused by hyperandrogenism in females. This potent anti-androgen inhibitor has been included in the Essential Medicines Lists of 55 countries.

Several synthesis methods of cyproterone acetate have been reported. Neumann et al. synthesized compound 4 as shown in FIG. 8a and derivatives thereof through an 18-step asymmetric synthesis starting from solasodine (Gynecological Endocrinology, 11, 1997, 411-433). European Patent No. 1359154A1 disclosed that Manosroi et al. improved the synthesis strategies for compound 4 starting from solasodine, developing 12-step, 14-step, and 16-step routes. Kongkathip et al. reported a 14-step stereoselective synthesis of 16β-chloro (or bromo)-4 starting from commercially available 16-dehydropregnenolone acetate (Synthetic Communications, 33, 2003, 1695-1706). These synthetic routes involve a large number of steps (12-18 steps), accompanied by waste generation and safety issues in handling hazardous materials and/or highly reactive substances/intermediates to achieve complex selective transformations, resulting in less than ideal overall efficiency of the reported batch methods.

Compared with batch synthesis, flow chemistry offers numerous advantages in the synthesis of organic small molecules, particularly in terms of environmental compatibility, safety, and synthetic efficiency, especially when considering scale-up production. These advantages have begun to have a significant impact across the chemical industry.

### SUMMARY

In view of the problems in the prior art, the disclosure provides a fully-continuous synthesis method of cyproterone acetate with high production efficiency, excellent product quality and fewer synthesis steps.

Technical solutions of the present disclosure are described as follows.

This application provides a fully-continuous synthesis method of cyproterone acetate. Starting from androstenedione, a 3-ketosteroid-Δ¹-dehydrogenase mutant ReM2 and other catalysts are used to catalyze the synthesis of androsta-1,4-diene-3,17-dione from androstenedione. Subsequent alkynylation and Rupe rearrangement yield Δ^{1,4,16}-triene-3,20-dione, which is then subjected to Δ⁶-dehydrogenation and C20-ketalization to obtain 17α-hydroxy-androsta-Δ^{1,4,16}-trien-3,20-dione 7. Then, a cascade reaction involving cyclopropanation, epoxidation, ring-opening, chlorination, dehydration, and deprotection is performed, followed by O-acetylation to obtain cyproterone acetate. Specifically, a fully continuous device composed of micromixers, microreactors, online solvent switching units, reagent recovery systems and low-boiling-point reagent feeding devices connected in series is adopted to realize online extraction, online solvent switching and online solvent concentration. The synthetic route is expressed as follows:

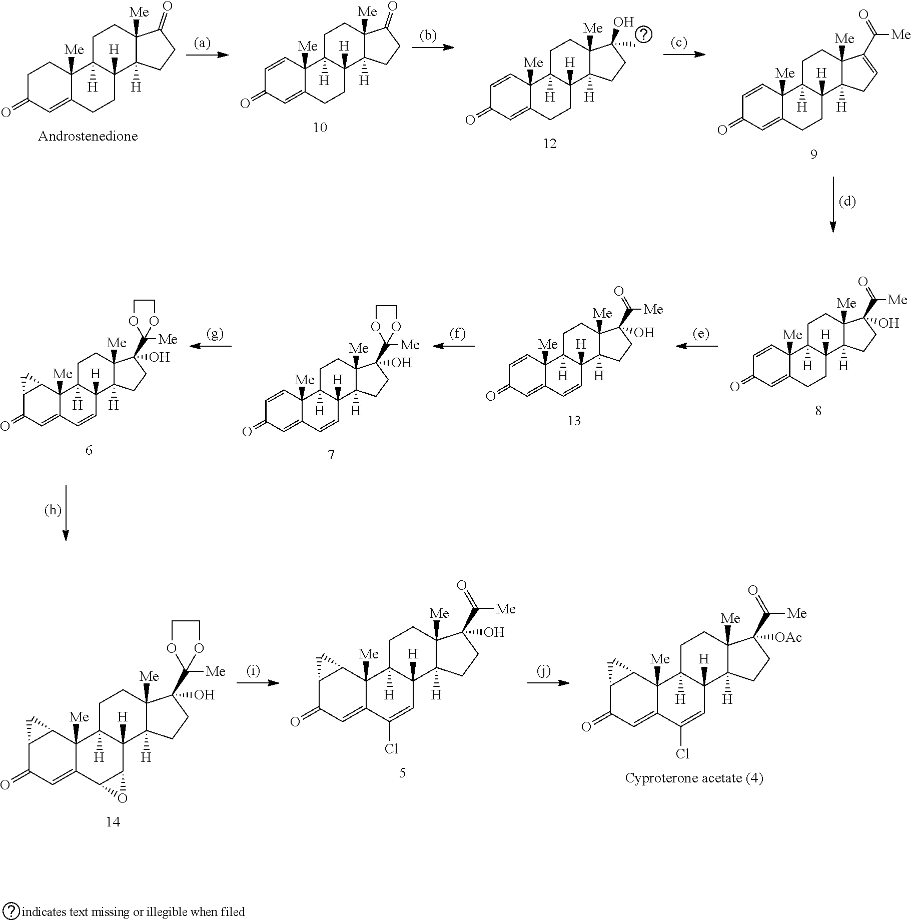

The fully-continuous synthesis method comprises:
(a) dissolving 4-androstene-3,17-dione and an electron acceptor in a first organic solvent to obtain a first feed liquid; and dispersing a first catalyst in a buffer solution to obtain a second feed liquid, wherein the first catalyst is an enzyme; and
pumping, by a first plunger pump, the first feed liquid and the second feed liquid into a first micromixer for mixing to obtain a first mixture, and conveying the first mixture to a dynamic tubular reactor and a first microreactor for Δ¹-dehydrogenation reaction to obtain a first reaction solution; subjecting the first reaction solution to extraction with an extraction solvent in an extraction device followed by phase separation in a first online gravity separation column and upper phase removal to collect a first lower phase; concentrating the first lower phase in a first concentration device to obtain a first concentrate; and loading the first concentrate onto a first stainless steel column filled with Na₂SO₄ and SiO₂ in a weight ratio of 1:1 for water and impurity removal to obtain an androstene-1,4-dien-3,17-dione (10)-containing mixed solution as a third feed liquid, as shown in FIG. 3.

In some embodiments, in step (a), a flow rate ratio of 4-androstene-3,17-dione to the electron acceptor to the first catalyst in the dynamic tubular reactor is controlled such that a molar ratio of the electron acceptor to 4-androstene-3,17-dione to the first catalyst is 0.1-0.3:1:1.0-5.0;
the electron acceptor is selected from the group consisting of phenazine methosulfate, 2,6-dichlorophenolindophenol, resazurin, Wurster's blue, methylene blue, coenzyme Q, vitamin K and menadione; preferably phenazine methosulfate;
the enzyme is derived from any one of 3-ketosteroid-Δ¹-dehydrogenases (Δ¹-KstDs) and their mutants from strains such as Arthrobacter simplex, Rhodococcus erythropolis, Mycobacterium neoaurum, and Mycobacterium smegmatis;
the first organic solvent is selected from the group consisting of dichloromethane, 1,2-dichloroethane, methanol, N,N-dimethylacetamide, dimethyl sulfoxide, tetrahydrofuran (THF) and 1,4-dioxane;
the buffer solution is selected from the group consisting of a phosphate buffer, a glycine-sodium hydroxide buffer, a Tris-hydrochloric acid buffer, a phthalic acid-hydrochloric acid buffer and a glycine-hydrochloric acid buffer;
the extraction solvent is dichloromethane or dichloroethane;
a temperature of the dynamic tubular reactor and a temperature of the first microreactor are each controlled at 20° C.-45° C.; a residence time of the first mixture in the dynamic tubular reactor is 30-120 min; and a residence time of the first mixture in the first microreactor is 22-90 min;
the extraction of the first reaction solution is performed for 1-5 min; and
the phase separation in the first online gravity separation column is performed for 3-20 min; and a residence time of the first lower phase in the first concentration device is 1-10 min.

The fully-continuous synthesis method further comprises:
(b) dissolving ethynylmagnesium bromide in a second organic solvent to obtain a fourth feed liquid; pumping, by a second plunger pump, the third feed liquid and the fourth feed liquid into a second micromixer for mixing to obtain a second mixture; conveying the second mixture to a second microreactor for alkynylation reaction to obtain a second reaction solution; and quenching the second reaction solution with an aqueous HCl solution in a third micromixer followed by extraction and phase separation in a second online gravity separation column to collect a second lower phase as a fifth feed liquid, wherein the second lower phase is a Δ^{1,4}-dien-3-one propargyl alcohol (12)-containing mixed solution, as shown in FIG. 4.

In some embodiments, in step (b), a molar concentration of ethynylmagnesium bromide is 100-300 mmol/L;
the second organic solvent is one or two selected from the group consisting of THF, N,N-dimethylacetamide (DMAC), dimethyl sulfoxide (DMSO) and hexamethylphosphoramide (HMPA); and
a temperature of the second microreactor is controlled at 20° C.-45° C.; and a residence time of the second mixture in the second microreactor is 30-120 min.

The fully-continuous synthesis method further comprises:
(c) dissolving an acidic reagent in dichloroethane to obtain a sixth feed liquid; pumping, by a third plunger pump, the fifth feed liquid and the sixth feed liquid into a fourth micromixer for mixing to obtain a third mixture; conveying the third mixture to a third microreactor for Rupe rearrangement to obtain a third reaction solution; and conveying the third reaction solution to a fifth micromixer through a first back pressure regulator, quenching the third reaction solution with a first aqueous NaHCO₃ solution in the fifth micromixer followed by extraction and phase separation in a third online gravity separation column to collect a third lower phase as a seventh feed liquid, wherein the third lower phase is a Δ^{1, 4, 16}-trien-3,20-dione (9)-containing mixed solution, as shown in FIG. 4.

In some embodiments, a molar concentration of the acidic reagent is 50-200 mmol/L;
the acidic reagent is selected from the group consisting of concentrated sulfuric acid, trifluoromethanesulfonic acid, an Eaton's reagent (MeSO₃H·P₂O₅), methanesulfonic acid, an Amberlyst 15 acidic resin, p-toluenesulfonic acid, aluminum trichloride and acetic acid; preferably the Eaton's reagent; and
a temperature of the third microreactor is controlled at 60° C.-100° C.; a molar ratio of the acidic reagent to Δ^{1, 4, 16}-trien-3,20-dione in the third lower phase is 1.5-3:1; and a residence time of the third mixture in the third microreactor is 20-30 min.

The fully-continuous synthesis method further comprises:
(d) dissolving a second catalyst and PhSiH₃ in a third organic solvent to obtain an eighth feed liquid; pumping, by a fourth plunger pump, the seventh feed liquid, the eighth feed liquid and oxygen into a sixth micromixer for mixing to obtain a fourth mixture, wherein the oxygen is stored in an oxygen cylinder with flow rate regulated by a check valve and a gas flow meter; conveying the fourth mixture to a fourth microreactor for Mukaiyama hydration reaction followed by mixing with an ethanol solution of P(OEt)₃ in a seventh micromixer to complete the Mukaiyama hydration reaction, so as to obtain a fourth reaction solution; quenching the fourth reaction solution with a first aqueous sodium chloride solution in an eighth micromixer followed by extraction and phase separation in a fourth online gravity separation column to collect a fourth lower phase; and mixing the fourth lower phase with a first N,N-dimethylformamide (DMF) solution in a ninth micromixer followed by concentration in a second concentration device to obtain a 17α-hydroxy-Δ^{1,4}-dien-3,20-dione (8)-containing mixed solution as a ninth feed liquid, as shown in FIG. 4.

In some embodiments, in step (d), a molar concentration of the second catalyst is 1-3 mol %;
the second catalyst is selected from the group consisting of Mn(dpm)₃, Co(acac)₂, Fe(dpm)₃, Fe(acac)₃, Fe(acac)₂, Co(acac)₃, cobalt acetate, cobalt (II) bromide, cobalt chloride and cobalt iso-octoate;
the third organic solvent is one or two selected from the group consisting of dichloromethane, 1,2-dichloroethane, methanol, ethanol and THF; and
a temperature of the fourth microreactor is controlled at 25° C.-35° C.; and a residence time of the fourth mixture in the fourth microreactor is 10-15 min.

The fully-continuous synthesis method further comprises:
(e) dissolving tetrachloro-p-benzoquinone (TCBQ) in a fourth organic solvent to obtain a tenth feed liquid; pumping, by a fifth plunger pump, the ninth feed liquid and the tenth feed liquid into a tenth micromixer for mixing to obtain a fifth mixture; conveying the fifth mixture to a fifth microreactor for Δ⁶-dehydrogenation reaction to obtain a fifth reaction solution; quenching the fifth reaction solution with a first 1 wt. % aqueous NaOH solution in an eleventh micromixer followed by extraction with DCM and phase separation in a fifth online gravity separation column to obtain a fifth lower phase and a first upper phase; subjecting the first upper phase to secondary extraction with DCM followed by phase separation in a sixth online gravity separation column to collect a sixth lower phase; mixing the fifth lower phase and the sixth lower phase with a second aqueous sodium chloride solution in a twelfth micromixer followed by phase separation in a seventh online gravity separation column to collect a seventh lower phase; and concentrating the seventh lower phase in a third concentration device followed by loading onto a second stainless steel column filled with Na₂SO₄ and SiO₂ in a weight ratio of 1:1 for moisture and impurity removal, so as to obtain a 17α-hydroxy-Δ^{1,4,6}-trien-3,20-dione (13)-containing dichloromethane solution as an eleventh feed liquid, as shown in FIG. 5.

In some embodiments, in step (e), the fourth organic solvent is selected from the group consisting of dichloromethane, 1,2-dichloroethane, DMAC, DMSO, 1,4-dioxane and tert-butanol; and a temperature of the fifth microreactor is controlled at 100° C.-140° C.; and a residence time of the fifth mixture in the fifth microreactor is 4-15 min.

The fully-continuous synthesis method further comprises:
(f) dissolving a third catalyst, ethylene glycol and a water absorbent in dichloromethane to obtain a twelfth feed liquid; mixing the eleventh feed liquid with the twelfth feed liquid in a thirteenth micromixer to obtain a sixth mixture; conveying the sixth mixture to a sixth microreactor for ketal protection reaction to obtain a sixth reaction solution; conveying the sixth reaction solution to a fourteenth micromixer through a second back pressure regulator, and quenching the sixth reaction solution with a second aqueous NaHCO₃ solution in the fourteenth micromixer followed by phase separation in an eighth online gravity separation column to collect an eighth lower phase; mixing the eighth lower phase with a second DMF solution in a fifteenth micromixer followed by concentration in a fourth concentration device to obtain a second concentrate; and loading the second concentrate onto a third stainless steel column filled with Na₂SO₄ and SiO₂ in a weight ratio of 1:1 for moisture and impurity removal, so as to obtain a 17α-hydroxy-Δ^{1,4,6}-trien-3-one-20-ketal (7)-containing mixed solution as a thirteenth feed liquid, as shown in FIG. 5.

In some embodiments, in step (f), the water absorbent is selected from the group consisting of a silica gel, a molecular sieve, anhydrous sodium sulfate, anhydrous magnesium sulfate, anhydrous calcium chloride and trimethyl orthoformate (TMOF);
the third catalyst is selected from the group consisting of concentrated sulfuric acid, p-toluenesulfonic acid, BF₃·Et₂O, TiCl₄, Bi(NO₃)₃, LiBF₄, Cu(BF₄)₂, RuCl₃·H₂O, CoCl₂, InCl₃, In(OTf)₃, ZrCl₄ and Zn(BF₄)₂; and a temperature of the sixth microreactor is controlled at 40° C.-100° C.; and a residence time of the sixth mixture in the sixth microreactor is 15-20 min.

The fully-continuous synthesis method further comprises:
(g) under a nitrogen atmosphere, dissolving trimethylsulfoxonium iodide (TMSOI) and a base in DMF followed by reaction at room temperature for 0.6-1.2 h to obtain a fourteenth feed liquid; mixing the thirteenth feed liquid with the fourteenth feed liquid in a sixteenth micromixer to obtain a seventh mixture; conveying the seventh mixture to a seventh microreactor for C1,C2 cyclopropanation reaction to obtain a seventh reaction solution; quenching the seventh reaction solution with a third aqueous sodium chloride solution in a seventeenth micromixer followed by extraction with dichloromethane and phase separation in a ninth online gravity separation column to obtain a ninth lower phase and a second upper phase; subjecting the second upper phase to secondary extraction with dichloromethane followed by phase separation in a tenth online gravity separation column to collect a tenth lower phase; and conveying the ninth lower phase and the tenth lower phase to a fifth concentration device for concentration, so as to obtain a 17α-hydroxy-1α,2α-cyclopropa-Δ^{4,6}-dien-3-one-20-ketal (6)-containing mixed solution as a fifteenth feed liquid, as shown in FIG. 6.

In some embodiments, in step (g), the base is selected from the group consisting of lithium bis(trimethylsilyl)amide (LiHMDS), 1,2-dianilinoethane (NODX), potassium tert-butoxide (^{t}BuOK), lithium diisopropylamide (LDA), sodium methoxide (MeONa) and NaH;
a temperature of the seventh microreactor is controlled at 25° C.-40° C.; and a residence time of the seventh mixture in the seventh microreactor is 15-20 min; and
a molar ratio of TMSOI to 17α-hydroxy-1α,2α-cyclopropa-Δ^{4,6}-dien-3-one-20-ketal in the fifteenth feed liquid is 2-3:1, and a molar ratio of the base to 17α-hydroxy-1α, 2α-cyclopropa-Δ^{4,6}-dien-3-one-20-ketal in the fifteenth feed liquid is 1.5-3.0:1.

The fully-continuous synthesis method further comprises:
(h) dissolving an oxidant in dichloromethane to obtain a sixteenth feed liquid; pumping, by a sixth plunger pump, the fifteenth feed liquid and the sixteenth feed liquid into an eighteenth micromixer for mixing to obtain an eighth mixture; conveying the eighth mixture to an eighth microreactor for C6, C7 epoxidation reaction to obtain an eighth reaction solution; conveying the eighth reaction solution to a nineteenth micromixer through a third back pressure regulator, and quenching the eighth reaction solution sequentially with an aqueous Na₂S₂O₃ solution in the nineteenth micromixer and a second 1 wt. % aqueous NaOH solution in a twentieth micromixer to remove residual oxidant followed by phase separation in an eleventh online gravity separation column to obtain an eleventh lower phase and a third upper phase; subjecting the third upper phase to secondary extraction with dichloromethane followed by phase separation in a twelfth online gravity separation column to collect a twelfth lower phase; and conveying the eleventh lower phase and the twelfth lower phase to a sixth concentration device for concentration, so as to obtain a 17α-hydroxy-6α,7α-epoxy-1α, 2α-cyclopropa-Δ⁴-en-3-one-20-ketal (14)-containing mixed solution as a seventeenth feed liquid, as shown in FIG. 7.

In some embodiments, in step (h), the oxidant is selected from the group consisting of performic acid, peracetic acid, perbenzoic acid, meta-chloroperoxybenzoic acid, trifluoroperacetic acid and hydrogen peroxide; and a temperature of the eighth microreactor is controlled at 25° C.-120° C.; and a residence time of the eighth mixture in the eighth microreactor is 10-20 min.

The fully-continuous synthesis method further comprises:
(i) dissolving N,N-dimethylacetamide hydrochloride (DMA·HCl) in DMF to obtain an eighteenth feed liquid; pumping, by a seventh plunger pump, the seventeenth feed liquid and the eighteenth feed liquid into a twenty-first micromixer for mixing to obtain a ninth mixture; conveying the ninth mixture to a ninth microreactor for C6,C7 epoxide ring-opening reaction, C6 chlorination, dehydration and deprotection cascade reaction to obtain a ninth reaction solution; conveying the ninth reaction solution to a twenty-second micromixer through a fourth back pressure regulator, and quenching the ninth reaction solution with a third 1 wt. % aqueous NaOH solution in the twenty-second micromixer followed by extraction with dichloromethane and phase separation in a thirteenth online gravity separation column to obtain a thirteenth lower phase and a fourth upper phase; subjecting the fourth upper phase to secondary extraction with dichloromethane followed by phase separation in a fourteenth online gravity separation column to collect a fourteenth lower phase; mixing the thirteenth lower phase and the fourteenth lower phase with a fourth aqueous sodium chloride solution in a twenty-third micromixer followed by phase separation in a fifteenth online gravity separation column to collect a fifteenth lower phase; and concentrating the fifteenth lower phase in a seventh concentration device followed by loading onto a fourth stainless steel column filled with Na₂SO₄ and SiO₂ in a weight ratio of 1:1 for moisture and impurity removal, so as to obtain a 17α-hydroxy-1α,2α-cyclopropa-6-chloro-Δ^{4,6}-dien-3,20-dione (5)-containing mixed solution as a nineteenth feed liquid, as shown in FIG. 7.

In some embodiments, in step (i), the steroidal C6,C7 epoxide ring-opening reaction, the C6 chlorination, the dehydration and the deprotection cascade reaction are performed as a one-pot reaction in the same reaction system; and a temperature of the ninth microreactor is controlled at 80° C.-180° C.; and a residence time of the ninth mixture in the ninth microreactor is 20-40 min.

The fully-continuous synthesis method further comprises:
(j) pumping, by an eighth plunger pump, the nineteenth feed liquid and an acetylation reagent into a twenty-fourth micromixer for mixing to obtain a tenth mixture; conveying the tenth mixture to a tenth microreactor containing an acid catalyst for acetylation reaction to obtain a tenth reaction solution; quenching the tenth reaction solution with a third aqueous NaHCO₃ solution in a twenty-fifth micromixer followed by phase separation in a sixteenth online gravity separation column to collect a fifth upper phase, wherein the fifth upper phase is a cyproterone acetate (4)-containing mixed solution; and
subjecting the fifth upper phase to chromatographic separation to obtain a pure cyproterone acetate product, as shown in FIG. 7.

In some embodiments, in step (j), the acetylation reagent is selected from the group consisting of acetic acid, acetic anhydride, acetyl chloride, trifluoroacetic anhydride and isopropenyl acetate; and the acid catalyst is selected from the group consisting of concentrated sulfuric acid, trifluoromethanesulfonic acid, methanesulfonic acid, an Amberlyst 15 acidic resin, p-toluenesulfonic acid and acetic acid.

In some embodiments, each of the first micromixer, the second micromixer, the third micromixer, the fourth micromixer, the fifth micromixer, the sixth micromixer, the seventh micromixer, the eighth micromixer, the ninth micromixer, the tenth micromixer, the eleventh micromixer, the twelfth micromixer, the thirteenth micromixer, the fourteenth micromixer, the fifteenth micromixer, the sixteenth micromixer, the seventeenth micromixer, the eighteenth micromixer, the nineteenth micromixer, the twentieth micromixer, the twenty-first micromixer, the twenty-second micromixer, the twenty-third micromixer, the twenty-fourth micromixer and the twenty-fifth micromixer is one of a T-type micromixer, a Y-type micromixer, a cross-type mixer, a coaxial flow micromixer and a flow-focusing micromixer;
each of the first microreactor, the second microreactor, the third microreactor, the fourth microreactor, the fifth microreactor, the sixth microreactor, the seventh microreactor, the eighth microreactor, the ninth microreactor and the tenth microreactor is a tubular microchannel reactor or a plate-type microchannel reactor;
each of the first concentration device, the second concentration device, the third concentration device, the fourth concentration device, the fifth concentration device, the sixth concentration device and the seventh concentration device is a tube-in-tube concentration device; and each of the first online gravity separation column, the second online gravity separation column, the third online gravity separation column, the fourth online gravity separation column, the fifth online gravity separation column, the sixth online gravity separation column, the seventh online gravity separation column, the eighth online gravity separation column, the ninth online gravity separation column, the tenth online gravity separation column, the eleventh online gravity separation column, the twelfth online gravity separation column, the thirteenth online gravity separation column, the fourteenth online gravity separation column, the fifteenth online gravity separation column and the sixteenth online gravity separation column adopts a glass tube gravity liquid-liquid separation device.

In some embodiments, a 3-ketosteroid 41-dehydrogenase mutant ReM2 enzyme or other 3-ketosteroid Δ¹-dehydrogenases is used to realize the continuous-flow enzymatic Δ¹-dehydrogenation of steroids.

The present disclosure adopts a combination of a dynamic tubular reactor (Nat Chem Eng 1, 522-531 (2024)) and a polytetrafluoroethylene reactor (refer to Chinese Patent Application Publication No. CN117695962A) to achieve the continuous-flow biocatalytic Δ¹-dehydrogenation of the substrate by ReM2 for synthesizing androstane-1,4-diene-3,17-dione, thereby solving the problem of pipeline clogging caused by steroid substrates.

In the present disclosure, as shown in FIG. 1, each of the first concentration device, the second concentration device, the third concentration device, the fourth concentration device, the fifth concentration device, the sixth concentration device and the seventh concentration device is composed of two feed liquid containers, a solvent storage tank, a recovery tank, a nitrogen cylinder, a concentration tube, a peristaltic pump, a hose, a T-type connector and a mass flow controller connected in series; the two feed liquid containers are connected via the T-type connector; the peristaltic pump is configured to pump a corresponding feed liquid into a polytetrafluoroethylene coil for mixing to obtain a corresponding mixture, so that the corresponding mixture enters the concentration tube along with N₂ via the T-type connector for heating and purging concentration to obtain a volatile solvent, the volatile solvent in the concentration tube enters a condenser to obtain a condensate, and the condensate enters the recovery tank to recover an evaporated solvent; and the peristaltic pump is configured such that after the corresponding feed liquid reaches a required concentration multiple, the corresponding feed liquid is transported by the peristaltic pump to a next operation stage.

In some embodiments, the first back pressure regulator, the second back pressure regulator, the third back pressure regulator and the fourth back pressure regulator are each made of stainless steel or Hastelloy; and the first back pressure regulator, the second back pressure regulator, the third back pressure regulator and the fourth back pressure regulator each have a connecting pipeline size of 1.6-20 mm and a pressure range of 0.1-2.0 MPa.

Compared to the prior art, the present disclosure has the following beneficial effects.

By reasonably combining micromixers, microreactors, and online post-reaction treatment devices according to the synthetic process characteristics of cyproterone acetate, the present disclosure achieves efficient and rapid synthesis of cyproterone acetate. Continuous synthesis of cyproterone acetate is realized through a self-designed solvent switching device, which significantly improves production efficiency. The synthetic device eliminates the need for isolation and purification of synthetic intermediates, effectively reduces the generation of three wastes (industrial wastewater, waste gas, and solid waste), lowers production costs, and causes little environmental pollution. Based on the innovative concept of the present disclosure, any other changes and modifications made by those skilled in the art without departing from the spirit of the application shall fall within the scope of this application defined by the appended claims.

### BRIEF DESCRIPTION OF THE DRAWINGS

FIG. 1 is a structural diagram of an online concentration device in accordance with an embodiment of the present disclosure;

FIG. 2 is a flow chart of a fully-continuous enzymatic-chemical synthesis method of cyproterone acetate in accordance with an embodiment of the present disclosure;

FIG. 3 is a structural diagram of a first module in accordance with an embodiment of the present disclosure, where PMS refers to phenazine methosulfate, OAR refers to dynamic tubular reactor, PTFE refers to polytetrafluoroethylene coil reactor, DCE refers to dichloroethane, DMF refers to N,N-dimethylformamide, and BPR refers to back pressure regulator;

FIG. 4 is a structural diagram of a second module in accordance with an embodiment of the present disclosure;

FIG. 5 is a structural diagram of a third module in accordance with an embodiment of the present disclosure;

FIG. 6 is a structural diagram of a fourth module in accordance with an embodiment of the present disclosure;

FIG. 7 is a structural diagram of a fifth module in accordance with an embodiment of the present disclosure; and

FIGS. 8a-c show proton nuclear magnetic resonance (¹H NMR) and carbon-13 nuclear magnetic resonance (¹³C NMR) spectra of a cyproterone acetate product in accordance with an embodiment of the present disclosure.

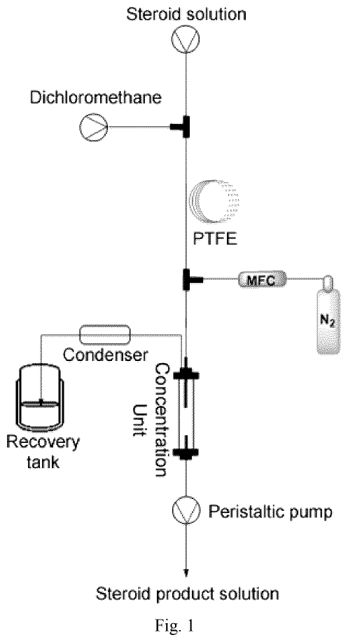

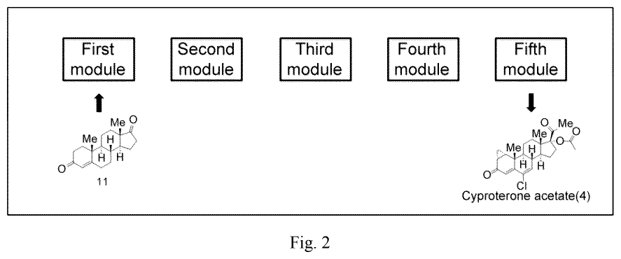

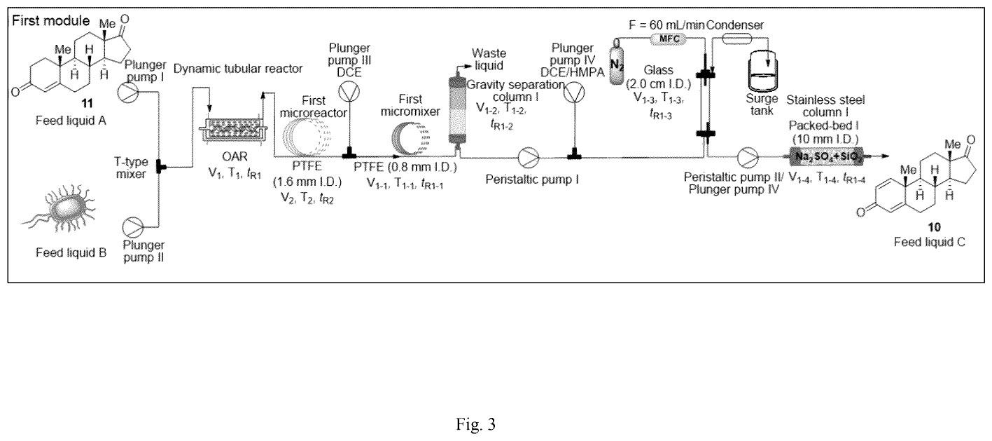

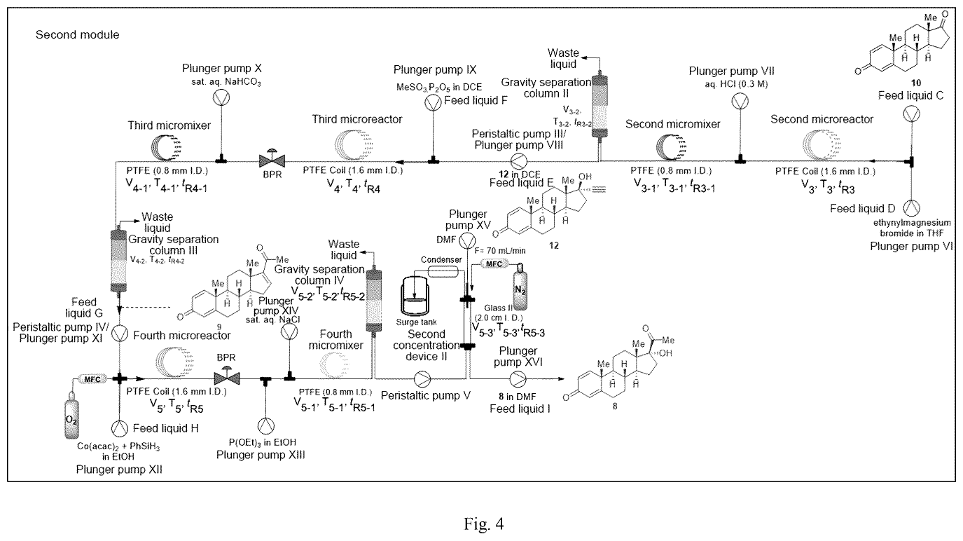

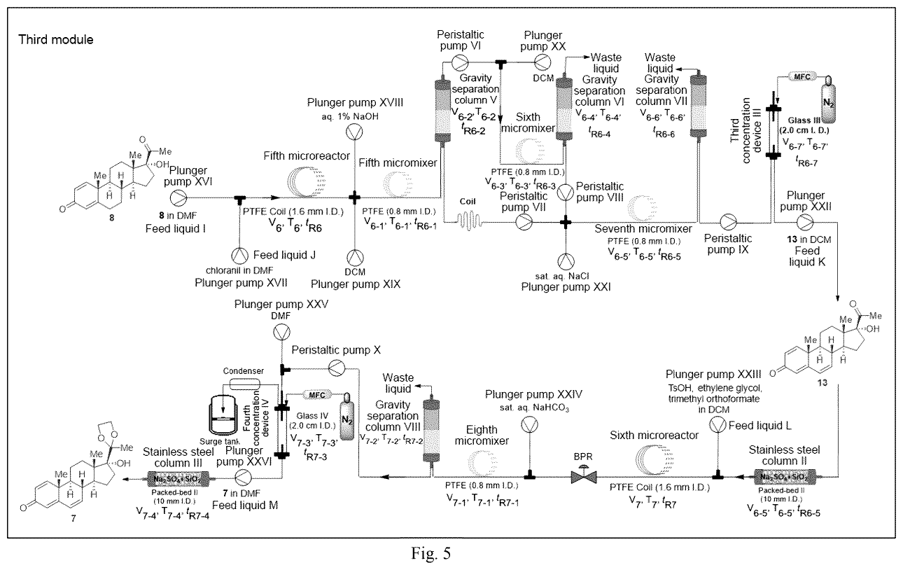

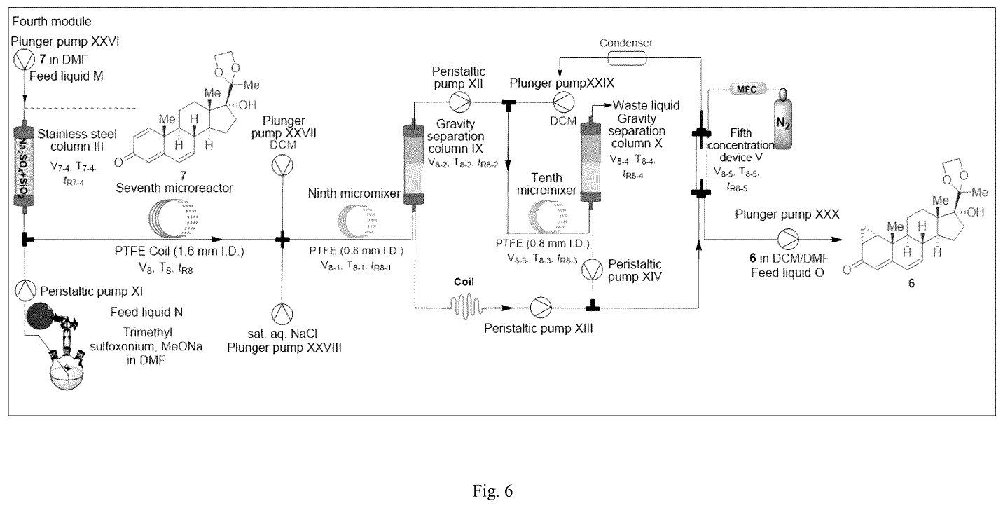

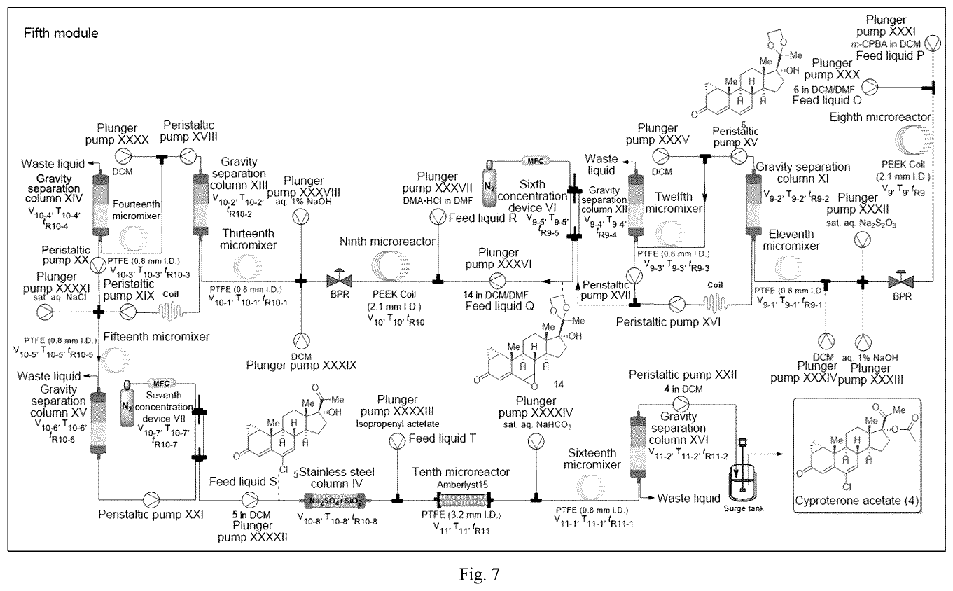

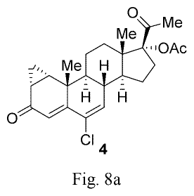

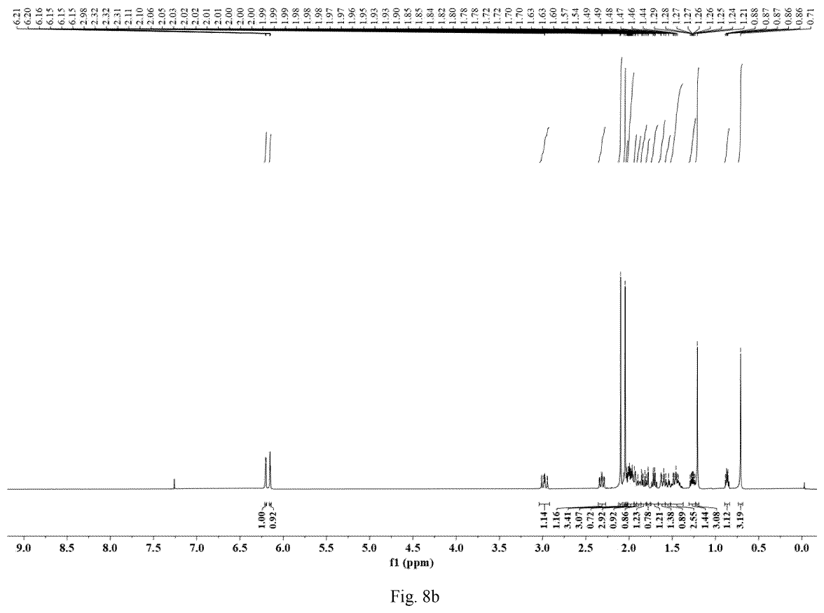

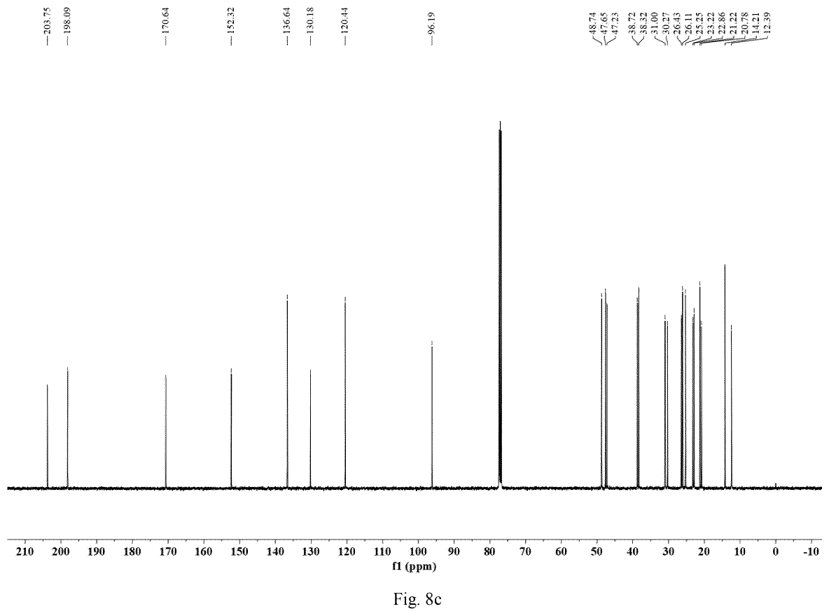

### DETAILED DESCRIPTION OF EMBODIMENTS

The present disclosure will be further described below the embodiments and the accompanying drawings.

### Example 1

Step (a) 4-androstene-3,17-dione (600 mM) and phenazine methosulfate (PMS, 60 mM, 0.1 equiv.) were dissolved in N,N-dimethylformamide (DMF, 50 mL, 0.1 mL/min) to obtain a feed liquid A with a flow rate of 0.1 mL/min. A 50.0 mg/mL ReM2 enzyme catalyst was dispersed in a Tris-HCl buffer (450 mL, 50 mM, pH 8.0, 0.9 mL/min) to obtain a feed liquid B. The feed liquid A, the feed liquid B and a O₂ gas (flow rate: 0.6 mL/min) were pumped into a first micromixer by a first plunger pump for mixing to obtain a first mixture. The first mixture was conveyed to a first dynamic tubular reactor and a first microreactor (V₁=30 mL) for reaction at 30° C. to obtain a first reaction solution, where the reaction was completed within a residence time of 10 min. The first reaction solution was subjected to phase separation in a first online gravity separation column (residence time t_{R1-2}=10 min) to collect a first lower phase (with a flow rate controlled by peristaltic pump: V=2 mL/min). The first lower phase and a mixed solution of dichloroethane (DCE) and hexamethylphosphoramide (HMPA) in a volume ratio of 9:1 (flow rate: 0.5 mL/min) were conveyed to a first concentration device for 5-fold concentration (flow rate: V=0.5 mL/min, temperature T=90° C., N₂ flow rate: 60 mL/min) to obtain a concentrate. The concentrate was loaded onto a first stainless steel column filled with Na₂SO₄ and SiO₂ in a weight ratio of 1:1 (residence time t_{R1-4}=40 min) for moisture and impurity trapping, so as to obtain a DCE solution containing a compound 10 as a feed liquid C. A total residence time was 82 min.

Step (b) Ethynylmagnesium bromide (0.5 M, 157.68 mM, 0.5 mL/min, 250 mL) was dissolved in tetrahydrofuran (THF) to obtain a feed liquid D. The feed liquid C and the feed liquid D were pumped into a second micromixer by a second plunger pump for mixing to obtain a second mixture. The second mixture was conveyed to a second microreactor (V₃=10 mL) for reaction at 60° C. to obtain a second reaction solution, where the reaction was completed within a residence time of 10 min. The second reaction solution was quenched with an aqueous HCl solution (0.3 M, 1.0 mL/min) in a third micromixer, then extracted in an extraction device (t_{R3-1}=1 min), and subjected to phase separation a second online gravity separation column (t_{R3-2}=10 min), so as to collect a second lower phase (with a flow rate controlled by a peristaltic pump: V=1.0 mL/min) as a feed liquid E. A total residence time was 21 min. The second lower phase was a DCE solution containing a compound 12.

Step (c) MeSO₃H·P₂O₅ (172.4 mM, 0.5 mL/min, 250 mL) was dissolved in DCE to obtain a feed liquid F. The feed liquid E (1.0 mL/min) and the feed liquid F were pumped into a fourth micromixer by a third plunger pump for mixing to obtain a third mixture. The third mixture was conveyed to a third microreactor (V₄=37.5 mL) for Rupe rearrangement at 80° C. under a back pressure of 5.0 bar with a residence time of 25 min to obtain a third reaction solution. The third reaction solution was quenched with an aqueous NaHCO₃ solution (2.0 mL/min) in a fifth micromixer, then extracted in the extraction device (t_{R4-1}=1 min), and subjected to phase separation in a third online gravity separation column (t_{R4-2}=10 min), so as to collect a third lower phase (with a flow rate controlled by a peristaltic pump: V=1.5 mL/min) as a feed liquid G. A total residence time was 36 min. The third lower phase was a DCE solution of a compound 9.

Step (d) Cobalt (II) acetylacetonate (Co(acac)₂, 1.97 mM) and phenylsilane (PhSiH₃, 43.68 mM) were dissolved in anhydrous ethanol (1.5 mL/min, 750 mL) to obtain a feed liquid H. The feed liquid G, the feed liquid H and an oxygen gas (1.0 mL/min) were pumped into a sixth micromixer by a fourth plunger pump for mixing to obtain a fourth mixture. The fourth mixture was pumped into a fourth microreactor for Mukaiyama hydration reaction (V₅=37.5 mL) at 35° C. under a back pressure of 2.0 bar with a residence time of 12.5 min, and then mixed with an ethanol solution of P(OEt)₃ (65.52 mM, 0.5 mL/min, 250 mL) in a seventh micromixer to complete the Mukaiyama hydration reaction, so as to obtain a fourth reaction solution. The fourth reaction solution was quenched with an aqueous NaCl solution (1.0 mL/min) in an eighth micromixer, then extracted in the extraction device (t_{R5-1}=1 min), and subjected to phase separation a fourth online gravity separation column (t_{R5-2}=10 min) to collect a fourth lower phase (with a flow rate controlled by a peristaltic pump: V=3.5 mL/min). The fourth lower phase was mixed with DMF (0.5 mL/min, 250 mL) in a ninth micromixer, and then fed into a second tube-in-tube concentration device for 7-fold concentration (V=0.5 mL/min, with parameters set as follows: temperature T=100° C., nitrogen (N₂) flow rate F=70 mL/min), so as to obtain an ethanol solution containing a compound 8 as a feed liquid I. A total residence time was 24.5 min.

Step (e) Tetrachloro-p-benzoquinone (123.12 mM) was dissolved in DMF (0.25 mL/min, 125 mL) to obtain a feed liquid J. The feed liquid I (0.5 mL/min) and the feed liquid J were pumped into a tenth micromixer for mixing to obtain a fifth mixture. The fifth mixture was conveyed to a fifth microreactor (V₆=3.0 mL) for 46-dehydrogenation reaction at 140° C. to obtain a fifth reaction solution. The 46-dehydrogenation reaction was completed within a residence time of 4 min. The fifth reaction solution was quenched with a 1 wt. % aqueous NaOH solution (2.0 mL/min, 1000 mL) in an eleventh micromixer, then extracted (t_{R6-1}=1 min) with DCM (2.0 mL/min, 1000 mL), and subjected to phase separation in a fifth online gravity separation column (t_{R6-2}=10 min) to obtain a fifth lower phase and a first upper phase. The fifth lower phase (with a flow rate controlled by a peristaltic pump: V=2.0 mL/min) was loaded onto a coil (20 mL) for buffering. The first upper phase (2.0 mL/min, 1000 mL) was subjected to secondary extraction in the extraction device (t_{R6-3}=1 min) followed by phase separation in a sixth online gravity separation column (t_{R6-4}=10 min) to collect a sixth lower phase (with a flow rate controlled by a peristaltic pump: V=2.0 mL/min). The fifth lower phase and the sixth lower phase was mixed with an aqueous NaCl solution in a twelfth micromixer, then washed in the extraction device (t_{R6-5}=1 min), and subjected to phase separation in a seventh online gravity separation column (t_{R6-6}=10 min) to collect a seventh lower phase (with a flow rate controlled by a peristaltic pump: V=4.0 mL/min). The seventh lower phase was conveyed to a third concentration device for 8-fold concentration (V=0.5 mL/min) at 70° C. with a nitrogen flow rate of 75 mL/min, and loaded onto a second stainless steel column filled with Na₂SO₄ and SiO₂ in a weight ratio of 1:1 for moisture and impurity removal, so as to obtain a DCM solution containing a compound 13 as a feed liquid K. A total residence time was 105 min.

Step (f) p-Toluenesulfonic acid (4.26 mM), ethylene glycol (191.7 mM) and trimethyl orthoformate (166.14 mM) were dissolved in DCM (0.5 mL/min, 250 mL) to obtain a feed liquid L. The feed liquid K (0.5 mL/min) and the feed liquid L were pumped into a thirteenth micromixer for mixing to obtain a sixth mixture. The sixth mixture was conveyed to a sixth microreactor (V₇=20 mL) for ketal protection reaction at 90° C. under a back pressure of 4.0 bar with a residence time of 20 min, so as to obtain a sixth reaction solution. The sixth reaction solution was subjected to online post-treatment, i.e., the sixth reaction solution was quenched with an aqueous NaHCO₃ solution (2.0 mL/min, 1000 mL) in a fourteenth micromixer, then extracted, and subjected to phase separation in an eighth online gravity separation column (t_{R7-2}=10 min) to collect an eighth lower phase (with a flow rate controlled by a peristaltic pump: V=1.0 mL/min). The eighth lower phase was mixed with DMF in a fifteenth micromixer, then subjected to 3-fold concentration (with parameters set as follows: V=0.5 mL/min, temperature T=45° C.; nitrogen flow rate F=35 mL/min) in a fourth concentration device to obtain a second concentrate. The second concentrate was loaded onto a third stainless steel column filled with Na₂SO₄ and SiO₂ in a weight ratio of 1:1 for moisture and impurity removal, so as to obtain a mixed DCM/DMF solution containing a compound 7 as a feed liquid M. A total residence time was 52 min.

Step (g) Under a nitrogen atmosphere, trimethylsulfoxonium iodide (TMSOI, 98 mM) and sodium methoxide (MeONa, 91.1 mM) were dissolved in DMF (0.5 mL/min, 250 mL) and pre-reacted in a batch reactor at room temperature for 1 h to obtain a feed liquid N. The feed liquid N (flow rate: 0.5 mL/min) was mixed with the feed liquid M in a sixteenth micromixer to obtain a seventh mixture. The seventh mixture was conveyed to a seventh microreactor (V₈=10 mL) for C1,C2 cyclopropanation reaction at 100° C. to obtain a seventh reaction solution. The C1, C2 cyclopropanation reaction was completed with a residence time of 10 min. The seventh reaction solution was quenched with an aqueous NaCl solution (1.0 mL/min, 500 mL) in a seventeenth micromixer, then extracted with DCM (2.0 mL/min, 1000 mL), and subjected to phase separation in a ninth online gravity separation column (t_{R8-2}=10 min) to obtain a ninth lower phase and a second upper phase. The ninth lower phase (with a flow rate controlled by a peristaltic pump: V=2.5 mL/min) was loaded onto a coil (10 mL) for buffering. The second upper phase was subjected to secondary extraction with DCM (1.5 mL/min, 750 mL) followed by phase separation in a tenth online gravity separation column (t_{R8-4}=3 min) to collect a tenth lower phase (with a flow rate controlled by a peristaltic pump: V=1.5 mL/min). The ninth lower phase and the tenth lower phase were conveyed to a fifth concentration device for 8-fold concentration (with parameters set as follows: V=0.5 mL/min, temperature T=75° C.; nitrogen flow rate F=70 mL/min), so as to obtain a mixed DCM/DMF solution containing a compound 6 as a feed liquid O. A total residence time was 46 min.

Step (h) M-chloroperoxybenzoic acid (m-CPBA, 94.9 mM) was dissolved in DCM (0.5 mL/min, 250 mL) to obtain a feed liquid P. The feed liquid O (0.5 mL/min) and the feed liquid P were pumped into an eighteenth micromixer by a sixth plunger pump for mixing to obtain an eighth mixture. The eighth mixture was conveyed to an eighth microreactor (V₉=10 mL) for C6, C7 epoxidation reaction at 110° C. under a back pressure of 6.0 bar with a residence time of 20 min, so as to obtain an eighth reaction solution. The eighth reaction solution was quenched with an aqueous Na₂S₂O₃ solution (1.0 mL/min, 500 mL) in a nineteenth micromixer to remove benzoic acid, and further quenched with a 1 wt. % aqueous NaOH solution (1.0 mL/min, 500 mL) in a twentieth micromixer to remove residual m-CPBA, followed by extraction (t_{R9-1}=1 min) and phase separation in an eleventh online gravity separation column (t_{R9-2}=10 min) to obtain an eleventh lower phase (with a flow rate controlled by a peristaltic pump: V=4.0 mL/min) and a third upper phase. The third upper phase was subjected to secondary extraction with DCM (2.0 mL/min, 1000 mL) followed by phase separation in a twelfth online gravity separation column (t_{R9-4}=2 min) to collect a twelfth lower phase (with a flow rate controlled by a peristaltic pump: V=2.0 mL/min). The eleventh lower phase and the twelfth lower phase were conveyed to a sixth concentration device for 6-fold concentration (with parameters set as follows: V=1.0 mL/min, T=60° C.; nitrogen flow rate F=60 mL/min), so as to obtain a DCM solution containing a compound 14 as a feed liquid Q. A total residence time was 35 min.

Step (i) N,N-dimethylacetamide hydrochloride (DMA·HCl, 177.13 mM) was dissolved in DMF (0.5 mL/min, 1000 mL) to obtain a feed liquid R. The feed liquid Q and the feed liquid R were pumped into a twenty-first micromixer by a seventh plunger pump for mixing to obtain a ninth mixture. The ninth mixture was conveyed to a ninth microreactor for C6,C7 epoxide ring-opening reaction, C6 chlorination, dehydration and deprotection cascade reaction at 180° C. under a back pressure of 16.0 bar with a residence time of 35 min, so as to obtain a ninth reaction solution. The ninth reaction solution was quenched with a 1 wt. % aqueous NaOH solution (1.0 mL/min, 500 mL) in a twenty-second micromixer, then extracted with DCM (2.0 mL/min, 1000 mL), and subjected to phase separation in a thirteenth online gravity separation column (t_{R10-2}=10 min) to obtain a thirteenth lower phase (with a flow rate controlled by a peristaltic pump: V=3.0 mL/min) and a fourth upper phase. The fourth upper phase was subjected to secondary extraction with DCM (2.0 mL/min, 1000 mL) followed by phase separation in a fourteenth online gravity separation column (t_{R10-4}=3 min) to collect a fourteenth lower phase (with a flow rate controlled by a peristaltic pump: V=2.0 mL/min). The thirteenth lower phase and the fourteenth lower phase were mixed with an aqueous NaCl solution in a twenty-third micromixer, and then subjected to phase separation in a fifteenth online gravity separation column (t_{R10-6}=10 min) to collect a fifteenth lower phase (with a flow rate controlled by a peristaltic pump: V=5.0 mL/min). The fifteenth lower phase was conveyed to a seventh concentration device for 10-fold concentration (with parameters set as follows: V=0.5 mL/min, T=100° C.; nitrogen flow rate F=90 mL/min), and then loaded onto a fourth stainless steel column filled with Na₂SO₄ and SiO₂ in a weight ratio of 1:1 for moisture and impurity removal, so as to obtain a DCM solution containing a compound 5 as a feed liquid S. A total residence time was 102 min.

Step (j) Isopropenyl acetate (750 mL, 1.5 mL/min) and the feed liquid S (0.5 mL/min) were pumped into a twenty-fourth micromixer by an eighth plunger pump for mixing to obtain a tenth mixture. The tenth mixture was conveyed to a tenth microreactor (V₁₁=30 mL) containing 3.5 g of Amberlyst 15 acidic resin for acetylation reaction at 40° C. with a residence time of 15 min, so as to obtain a tenth reaction solution. The tenth reaction solution was quenched with a saturated aqueous NaHCO₃ solution (1.0 mL/min, 500 mL) in a twenty-fifth micromixer, then subjected to phase separation in a sixteenth online gravity separation column (t_{R11-2}=10 min) to collect a fifth upper phase (with a flow rate controlled by a peristaltic pump: V=2.0 mL/min). The fifth upper phase was a mixed DCM/isopropenyl acetate solution containing a compound 4. A total residence time was 27 min.

### Example 2

Step (a) 4-androstene-3,17-dione (600 mM) and methylene blue (PMS, 60 mM, 0.1 equiv.) were dissolved in DMF (50 mL, 0.1 mL/min) to obtain a feed liquid A with a flow rate of 0.1 mL/min. A 50.0 mg/mL ReM2 enzyme catalyst was dispersed in a Tris-HCl buffer (450 mL, 50 mM, pH 8.0, 0.9 mL/min) to obtain a feed liquid B. The feed liquid A, the feed liquid B and a O₂ gas (flow rate: 0.6 mL/min) were pumped into a first micromixer by a first plunger pump for mixing to obtain a first mixture. The first mixture was conveyed to a first dynamic tubular reactor and a first microreactor (V₁=30 mL) for reaction at 40° C. to obtain a first reaction solution, where the reaction was completed within a residence time of 10 min. The first reaction solution was subjected to phase separation in a first online gravity separation column (residence time t_{R1-2}=10 min) to collect a first lower phase (with a flow rate controlled by peristaltic pump: V=2 mL/min). The first lower phase and a mixed solution of DCE and HMPA in a volume ratio of 9:1 (flow rate: 0.5 mL/min) were conveyed to a first concentration device for 5-fold concentration (flow rate: V=0.5 mL/min, temperature T=90° C., N₂ flow rate: 60 mL/min) to obtain a concentrate. The concentrate was loaded onto a first stainless steel column filled with Na₂SO₄ and SiO₂ in a weight ratio of 1:1 (residence time t_{R1-4}=40 min) for moisture and impurity trapping, so as to obtain a DCE solution containing a compound 10 as a feed liquid C. A total residence time was 82 min.

Step (b) Ethynylmagnesium bromide (0.5 M, 157.68 mM, 0.5 mL/min, 250 mL) was dissolved in THF to obtain a feed liquid D. The feed liquid C and the feed liquid D were pumped into a second micromixer by a second plunger pump for mixing to obtain a second mixture. The second mixture was conveyed to a second microreactor (V₃=10 mL) for reaction at 50° C. to obtain a second reaction solution, where the reaction was completed within a residence time of 10 min. The second reaction solution was quenched with an aqueous HCl solution (0.3 M, 1.0 mL/min) in a third micromixer, then extracted in an extraction device (t_{R3-1}=1 min), and subjected to phase separation a second online gravity separation column (t_{R3-2}=10 min), so as to collect a second lower phase (with a flow rate controlled by a peristaltic pump: V=1.0 mL/min) as a feed liquid E. A total residence time was 21 min. The second lower phase was a DCE solution containing a compound 12.

Step (c) MeSO₃H·P₂O₅ (172.4 mM, 0.5 mL/min, 250 mL) was dissolved in DCE to obtain a feed liquid F. The feed liquid E (1.0 mL/min) and the feed liquid F were pumped into a fourth micromixer by a third plunger pump for mixing to obtain a third mixture. The third mixture was conveyed to a third microreactor (V₄=37.5 mL) for Rupe rearrangement at 80° C. under a back pressure of 5.0 bar with a residence time of 25 min to obtain a third reaction solution. The third reaction solution was quenched with an aqueous NaHCO₃ solution (2.0 mL/min) in a fifth micromixer, then extracted in the extraction device (t_{R4-1}=1 min), and subjected to phase separation in a third online gravity separation column (t_{R4-2}=10 min), so as to collect a third lower phase (with a flow rate controlled by a peristaltic pump: V=1.5 mL/min) as a feed liquid G. A total residence time was 36 min. The third lower phase was a DCE solution of a compound 9.

Step (d) Co(acac)₂ (1.97 mM) and PhSiH₃ (43.68 mM) were dissolved in anhydrous ethanol (1.5 mL/min, 750 mL) to obtain a feed liquid H. The feed liquid G, the feed liquid H and an oxygen gas (1.0 mL/min) were pumped into a sixth micromixer by a fourth plunger pump for mixing to obtain a fourth mixture. The fourth mixture was pumped into a fourth microreactor for Mukaiyama hydration reaction (V₅=37.5 mL) at 35° C. under a back pressure of 2.0 bar with a residence time of 15 min, and then mixed with an ethanol solution of P(OEt)₃ (65.52 mM, 0.5 mL/min, 250 mL) in a seventh micromixer to complete the Mukaiyama hydration reaction, so as to obtain a fourth reaction solution. The fourth reaction solution was quenched with an aqueous NaCl solution (1.0 mL/min) in an eighth micromixer, then extracted in the extraction device (t_{R5-1}=1 min), and subjected to phase separation a fourth online gravity separation column (t_{R5-2}=10 min) to collect a fourth lower phase (with a flow rate controlled by a peristaltic pump: V=3.5 mL/min). The fourth lower phase was mixed with DMF (0.5 mL/min, 250 mL) in a ninth micromixer, and then fed into a second tube-in-tube concentration device for 7-fold concentration (V=0.5 mL/min, with parameters set as follows: temperature T=100° C., nitrogen (N₂) flow rate F=70 mL/min), so as to obtain an ethanol solution containing a compound 8 as a feed liquid I. A total residence time was 27 min.

Step (e) Tetrachloro-p-benzoquinone (123.12 mM) was dissolved in DMF (0.25 mL/min, 125 mL) to obtain a feed liquid J. The feed liquid I (0.5 mL/min) and the feed liquid J were pumped into a tenth micromixer for mixing to obtain a fifth mixture. The fifth mixture was conveyed to a fifth microreactor (V₆=3.0 mL) for 46-dehydrogenation reaction at 120° C. to obtain a fifth reaction solution. The 46-dehydrogenation reaction was completed within a residence time of 15 min. The fifth reaction solution was quenched with a 1 wt. % aqueous NaOH solution (2.0 mL/min, 1000 mL) in an eleventh micromixer, then extracted (t_{R6-1}=1 min) with DCM (2.0 mL/min, 1000 mL), and subjected to phase separation in a fifth online gravity separation column (t_{R6-2}=10 min) to obtain a fifth lower phase and a first upper phase. The fifth lower phase (with a flow rate controlled by a peristaltic pump: V=2.0 mL/min) was loaded onto a coil (20 mL) for buffering. The first upper phase (2.0 mL/min, 1000 mL) was subjected to secondary extraction in the extraction device (t_{R6-3}=1 min) followed by phase separation in a sixth online gravity separation column (t_{R6-4}=10 min) to collect a sixth lower phase (with a flow rate controlled by a peristaltic pump: V=2.0 mL/min). The fifth lower phase and the sixth lower phase was mixed with an aqueous NaCl solution in a twelfth micromixer, then washed in the extraction device (t_{R6-5}=1 min), and subjected to phase separation in a seventh online gravity separation column (t_{R6-6}=10 min) to collect a seventh lower phase (with a flow rate controlled by a peristaltic pump: V=4.0 mL/min). The seventh lower phase was conveyed to a third concentration device for 8-fold concentration (V=0.5 mL/min) at 70° C. with a nitrogen flow rate of 75 mL/min, and loaded onto a second stainless steel column filled with Na₂SO₄ and SiO₂ in a weight ratio of 1:1 for moisture and impurity removal, so as to obtain a DCM solution containing a compound 13 as a feed liquid K. A total residence time was 116 min.

Step (f) p-Toluenesulfonic acid (4.26 mM), ethylene glycol (191.7 mM) and trimethyl orthoformate (166.14 mM) were dissolved in DCM (0.5 mL/min, 250 mL) to obtain a feed liquid L. The feed liquid K (0.5 mL/min) and the feed liquid L were pumped into a thirteenth micromixer for mixing to obtain a sixth mixture. The sixth mixture was conveyed to a sixth microreactor (V₇=20 mL) for ketal protection reaction at 90° C. under a back pressure of 6.0 bar with a residence time of 10 min, so as to obtain a sixth reaction solution. The sixth reaction solution was subjected to online post-treatment, i.e., the sixth reaction solution was quenched with an aqueous NaHCO₃ solution (2.0 mL/min, 1000 mL) in a fourteenth micromixer, then extracted, and subjected to phase separation in an eighth online gravity separation column (t_{R7-2}=10 min) to collect an eighth lower phase (with a flow rate controlled by a peristaltic pump: V=1.0 mL/min). The eighth lower phase was mixed with DMF in a fifteenth micromixer, then subjected to 3-fold concentration (with parameters set as follows: V=0.5 mL/min, temperature T=45° C.; nitrogen flow rate F=35 mL/min) in a fourth concentration device to obtain a second concentrate. The second concentrate was loaded onto a third stainless steel column filled with Na₂SO₄ and SiO₂ in a weight ratio of 1:1 for moisture and impurity removal, so as to obtain a mixed DCM/DMF solution containing a compound 7 as a feed liquid M. A total residence time was 42 min.

Step (g) Under a nitrogen atmosphere, TMSOI (98 mM) and MeONa (91.1 mM) were dissolved in DMF (0.5 mL/min, 250 mL) and pre-reacted in a batch reactor at room temperature for 1 h to obtain a feed liquid N. The feed liquid N (flow rate: 0.5 mL/min) was mixed with the feed liquid M in a sixteenth micromixer to obtain a seventh mixture. The seventh mixture was conveyed to a seventh microreactor (V₈=10 mL) for C1,C2 cyclopropanation reaction at 100° C. to obtain a seventh reaction solution. The C1, C2 cyclopropanation reaction was completed with a residence time of 10 min. The seventh reaction solution was quenched with an aqueous NaCl solution (1.0 mL/min, 500 mL) in a seventeenth micromixer, then extracted with DCM (2.0 mL/min, 1000 mL), and subjected to phase separation in a ninth online gravity separation column (t_{R8-2}=10 min) to obtain a ninth lower phase and a second upper phase. The ninth lower phase (with a flow rate controlled by a peristaltic pump: V=2.5 mL/min) was loaded onto a coil (10 mL) for buffering. The second upper phase was subjected to secondary extraction with DCM (1.5 mL/min, 750 mL) followed by phase separation in a tenth online gravity separation column (t_{R8-4}=3 min) to collect a tenth lower phase (with a flow rate controlled by a peristaltic pump: V=1.5 mL/min). The ninth lower phase and the tenth lower phase were conveyed to a fifth concentration device for 8-fold concentration (with parameters set as follows: V=0.5 mL/min, temperature T=75° C.; nitrogen flow rate F=70 mL/min), so as to obtain a mixed DCM/DMF solution containing a compound 6 as a feed liquid O. A total residence time was 46 min.

Step (h) m-CPBA (94.9 mM) was dissolved in DCM/DMF (0.5 mL/min, 250 mL) to obtain a feed liquid P. The feed liquid O (0.5 mL/min) and the feed liquid P were pumped into an eighteenth micromixer by a sixth plunger pump for mixing to obtain an eighth mixture. The eighth mixture was conveyed to an eighth microreactor (V₉=10 mL) for C6, C7 epoxidation reaction at 110° C. under a back pressure of 6.0 bar with a residence time of 20 min, so as to obtain an eighth reaction solution. The eighth reaction solution was quenched with an aqueous Na₂S₂O₃ solution (1.0 mL/min, 500 mL) in a nineteenth micromixer to remove benzoic acid, and further quenched with a 1 wt. % aqueous NaOH solution (1.0 mL/min, 500 mL) in a twentieth micromixer to remove residual m-CPBA, followed by extraction (t_{R9-1}=1 min) and phase separation in an eleventh online gravity separation column (t_{R9-2}=10 min) to obtain an eleventh lower phase (with a flow rate controlled by a peristaltic pump: V=4.0 mL/min) and a third upper phase. The third upper phase was subjected to secondary extraction with DCM (2.0 mL/min, 1000 mL) followed by phase separation in a twelfth online gravity separation column (t_{R9-4}=2 min) to collect a twelfth lower phase (with a flow rate controlled by a peristaltic pump: V=2.0 mL/min). The eleventh lower phase and the twelfth lower phase were conveyed to a sixth concentration device for 6-fold concentration (with parameters set as follows: V=1.0 mL/min, T=60° C.; nitrogen flow rate F=60 mL/min), so as to obtain a DCM solution containing a compound 14 as a feed liquid Q. A total residence time was 35 min.

Step (i) DMA HCl (177.13 mM) was dissolved in DMF (0.5 mL/min, 1000 mL) to obtain a feed liquid R. The feed liquid Q and the feed liquid R were pumped into a twenty-first micromixer by a seventh plunger pump for mixing to obtain a ninth mixture. The ninth mixture was conveyed to a ninth microreactor for C6,C7 epoxide ring-opening reaction, C6 chlorination, dehydration and deprotection cascade reaction at 180° C. under a back pressure of 16.0 bar with a residence time of 35 min, so as to obtain a ninth reaction solution. The ninth reaction solution was quenched with a 1 wt. % aqueous NaOH solution (1.0 mL/min, 500 mL) in a twenty-second micromixer, then extracted with DCM (2.0 mL/min, 1000 mL), and subjected to phase separation in a thirteenth online gravity separation column (t_{R10-2}=10 min) to obtain a thirteenth lower phase (with a flow rate controlled by a peristaltic pump: V=3.0 mL/min) and a fourth upper phase. The fourth upper phase was subjected to secondary extraction with DCM (2.0 mL/min, 1000 mL) followed by phase separation in a fourteenth online gravity separation column (t_{R10-4}=3 min) to collect a fourteenth lower phase (with a flow rate controlled by a peristaltic pump: V=2.0 mL/min). The thirteenth lower phase and the fourteenth lower phase were mixed with an aqueous NaCl solution in a twenty-third micromixer, and then subjected to phase separation in a fifteenth online gravity separation column (t_{R10-6}=10 min) to collect a fifteenth lower phase (with a flow rate controlled by a peristaltic pump: V=5.0 mL/min). The fifteenth lower phase was conveyed to a seventh concentration device for 10-fold concentration (with parameters set as follows: V=0.5 mL/min, T=100° C.; nitrogen flow rate F=90 mL/min), and then loaded onto a fourth stainless steel column filled with Na₂SO₄ and SiO₂ in a weight ratio of 1:1 for moisture and impurity removal, so as to obtain a DCM solution containing a compound 5 as a feed liquid S. A total residence time was 102 min.

Step (j) Isopropenyl acetate (750 mL, 1.5 mL/min) and the feed liquid S (0.5 mL/min) were pumped into a twenty-fourth micromixer by an eighth plunger pump for mixing to obtain a tenth mixture. The tenth mixture was conveyed to a tenth microreactor (V₁₁=30 mL) containing 3 g of Amberlyst 15 acidic resin for acetylation reaction at 40° C. with a residence time of 20 min, so as to obtain a tenth reaction solution. The tenth reaction solution was quenched with a saturated aqueous NaHCO₃ solution (1.0 mL/min, 500 mL) in a twenty-fifth micromixer, then subjected to phase separation in a sixteenth online gravity separation column (t_{R11-2}=10 min) to collect a fifth upper phase (with a flow rate controlled by a peristaltic pump: V=2.0 mL/min). The fifth upper phase was a mixed DCM/isopropenyl acetate solution containing a compound 4. A total residence time was 32 min.

### Example 3

Step (a) 4-androstene-3,17-dione (600 mM) and 2,6-dichlorophenolindophenol (60 mM, 0.1 equiv.) were dissolved in DMF (50 mL, 0.1 mL/min) to obtain a feed liquid A with a flow rate of 0.1 mL/min. A 60.0 mg/mL ReM2 enzyme catalyst was dispersed in a Tris-HCl buffer (450 mL, 50 mM, pH 8.0, 0.9 mL/min) to obtain a feed liquid B. The feed liquid A, the feed liquid B and a O₂ gas (flow rate: 0.6 mL/min) were pumped into a first micromixer by a first plunger pump for mixing to obtain a first mixture. The first mixture was conveyed to a first dynamic tubular reactor and a first microreactor (V₁=30 mL) for reaction at 35° C. to obtain a first reaction solution, where the reaction was completed within a residence time of 6 min. The first reaction solution was subjected to phase separation in a first online gravity separation column (residence time t_{R1-2}=10 min) to collect a first lower phase (with a flow rate controlled by peristaltic pump: V=2 mL/min). The first lower phase was conveyed to a first concentration device for 5-fold concentration (flow rate: V=0.5 mL/min, temperature T=90° C., N₂ flow rate: 60 mL/min) to obtain a concentrate. The concentrate was loaded onto a first stainless steel column filled with Na₂SO₄ and SiO₂ in a weight ratio of 1:1 (residence time t_{R1-4}=40 min) for moisture and impurity trapping, so as to obtain a DCE solution containing a compound 10 as a feed liquid C. A total residence time was 78 min.

Step (b) Ethynylmagnesium bromide (0.5 M, 157.68 mM, 0.5 mL/min, 250 mL) was dissolved in THF to obtain a feed liquid D. The feed liquid C and the feed liquid D were pumped into a second micromixer by a second plunger pump for mixing to obtain a second mixture. The second mixture was conveyed to a second microreactor (V₃=10 mL) for reaction at 50° C. to obtain a second reaction solution, where the reaction was completed within a residence time of 10 min. The second reaction solution was quenched with an aqueous HCl solution (0.3 M, 1.0 mL/min) in a third micromixer, then extracted in an extraction device (t_{R3-1}=1 min), and subjected to phase separation a second online gravity separation column (t_{R3-2}=10 min), so as to collect a second lower phase (with a flow rate controlled by a peristaltic pump: V=1.0 mL/min) as a feed liquid E. A total residence time was 21 min. The second lower phase was a DCE solution containing a compound 12.

Step (c) An Eaton's reagent (172.4 mM, 0.5 mL/min, 250 mL) was dissolved in DCE to obtain a feed liquid F. The feed liquid E (1.0 mL/min) and the feed liquid F were pumped into a fourth micromixer by a third plunger pump for mixing to obtain a third mixture. The third mixture was conveyed to a third microreactor (V₄=37.5 mL) for Rupe rearrangement at 80° C. under a back pressure of 6.0 bar with a residence time of 30 min to obtain a third reaction solution. The third reaction solution was quenched with an aqueous NaHCO₃ solution (2.0 mL/min) in a fifth micromixer, then extracted in the extraction device (t_{R4-1}=1 min), and subjected to phase separation in a third online gravity separation column (t_{R4-2}=10 min), so as to collect a third lower phase (with a flow rate controlled by a peristaltic pump: V=1.5 mL/min) as a feed liquid G. A total residence time was 41 min. The third lower phase was a DCE solution of a compound 9.

Step (d) Co(acac)₂ (1.97 mM) and PhSiH₃ (43.68 mM) were dissolved in anhydrous ethanol (1.5 mL/min, 750 mL) to obtain a feed liquid H. The feed liquid G, the feed liquid H and an oxygen gas (1.0 mL/min) were pumped into a sixth micromixer by a fourth plunger pump for mixing to obtain a fourth mixture. The fourth mixture was pumped into a fourth microreactor for Mukaiyama hydration reaction (V₅=37.5 mL) at 35° C. under a back pressure of 2.0 bar with a residence time of 15 min, and then mixed with an ethanol solution of P(OEt)₃ (65.52 mM, 0.5 mL/min, 250 mL) in a seventh micromixer to complete the Mukaiyama hydration reaction, so as to obtain a fourth reaction solution. The fourth reaction solution was quenched with an aqueous NaCl solution (1.0 mL/min) in an eighth micromixer, then extracted in the extraction device (t_{R5-1}=1 min), and subjected to phase separation a fourth online gravity separation column (t_{R5-2}=10 min) to collect a fourth lower phase (with a flow rate controlled by a peristaltic pump: V=3.5 mL/min). The fourth lower phase was mixed with DMF (0.5 mL/min, 250 mL) in a ninth micromixer, and then fed into a second tube-in-tube concentration device for 7-fold concentration (V=0.5 mL/min, with parameters set as follows: temperature T=100° C., nitrogen (N₂) flow rate F=70 mL/min), so as to obtain an ethanol solution containing a compound 8 as a feed liquid I. A total residence time was 27 min.

Step (e) Tetrachloro-p-benzoquinone (123.12 mM) was dissolved in DMF (0.25 mL/min, 125 mL) to obtain a feed liquid J. The feed liquid I (0.5 mL/min) and the feed liquid J were pumped into a tenth micromixer for mixing to obtain a fifth mixture. The fifth mixture was conveyed to a fifth microreactor (V₆=3.0 mL) for 46-dehydrogenation reaction at 120° C. to obtain a fifth reaction solution. The 46-dehydrogenation reaction was completed within a residence time of 15 min. The fifth reaction solution was quenched with a 1 wt. % aqueous NaOH solution (2.0 mL/min, 1000 mL) in an eleventh micromixer, then extracted (t_{R6-1}=1 min) with DCM (2.0 mL/min, 1000 mL), and subjected to phase separation in a fifth online gravity separation column (t_{R6-2}=10 min) to obtain a fifth lower phase and a first upper phase. The fifth lower phase (with a flow rate controlled by a peristaltic pump: V=2.0 mL/min) was loaded onto a coil (20 mL) for buffering. The first upper phase (2.0 mL/min, 1000 mL) was subjected to secondary extraction in the extraction device (t_{R6-3}=1 min) followed by phase separation in a sixth online gravity separation column (t_{R6-4}=10 min) to collect a sixth lower phase (with a flow rate controlled by a peristaltic pump: V=2.0 mL/min). The fifth lower phase and the sixth lower phase was mixed with an aqueous NaCl solution in a twelfth micromixer, then washed in the extraction device (t_{R6-5}=1 min), and subjected to phase separation in a seventh online gravity separation column (t_{R6-6}=10 min) to collect a seventh lower phase (with a flow rate controlled by a peristaltic pump: V=4.0 mL/min). The seventh lower phase was conveyed to a third concentration device for 8-fold concentration (V=0.5 mL/min) at 70° C. with a nitrogen flow rate of 75 mL/min, and loaded onto a second stainless steel column filled with Na₂SO₄ and SiO₂ in a weight ratio of 1:1 for moisture and impurity removal, so as to obtain a DCM solution containing a compound 13 as a feed liquid K. A total residence time was 116 min.

Step (f) p-Toluenesulfonic acid (4.26 mM), ethylene glycol (191.7 mM) and trimethyl orthoformate (166.14 mM) were dissolved in DCM (0.5 mL/min, 250 mL) to obtain a feed liquid L. The feed liquid K (0.5 mL/min) and the feed liquid L were pumped into a thirteenth micromixer for mixing to obtain a sixth mixture. The sixth mixture was conveyed to a sixth microreactor (V₇=20 mL) for ketal protection reaction at 80° C. under a back pressure of 6.0 bar with a residence time of 10 min, so as to obtain a sixth reaction solution. The sixth reaction solution was subjected to online post-treatment, i.e., the sixth reaction solution was quenched with an aqueous NaHCO₃ solution (2.0 mL/min, 1000 mL) in a fourteenth micromixer, then extracted, and subjected to phase separation in an eighth online gravity separation column (t_{R7-2}=10 min) to collect an eighth lower phase (with a flow rate controlled by a peristaltic pump: V=1.0 mL/min). The eighth lower phase was mixed with DMF in a fifteenth micromixer, then subjected to 3-fold concentration (with parameters set as follows: V=0.5 mL/min, temperature T=45° C.; nitrogen flow rate F=35 mL/min) in a fourth concentration device to obtain a second concentrate. The second concentrate was loaded onto a third stainless steel column filled with Na₂SO₄ and SiO₂ in a weight ratio of 1:1 for moisture and impurity removal, so as to obtain a mixed DCM/DMF solution containing a compound 7 as a feed liquid M. A total residence time was 42 min.

Step (g) Under a nitrogen atmosphere, TMSOI (98 mM) and MeONa (91.1 mM) were dissolved in DMF (0.5 mL/min, 250 mL) and pre-reacted in a batch reactor at room temperature for 1 h to obtain a feed liquid N. The feed liquid N (flow rate: 0.5 mL/min) was mixed with the feed liquid M in a sixteenth micromixer to obtain a seventh mixture. The seventh mixture was conveyed to a seventh microreactor (V₈=10 mL) for C1,C2 cyclopropanation reaction at 120° C. to obtain a seventh reaction solution. The C1, C2 cyclopropanation reaction was completed with a residence time of 7.5 min. The seventh reaction solution was quenched with an aqueous NaCl solution (1.0 mL/min, 500 mL) in a seventeenth micromixer, then extracted with DCM (2.0 mL/min, 1000 mL), and subjected to phase separation in a ninth online gravity separation column (t_{R8-2}=10 min) to obtain a ninth lower phase and a second upper phase. The ninth lower phase (with a flow rate controlled by a peristaltic pump: V=2.5 mL/min) was loaded onto a coil (10 mL) for buffering. The second upper phase was subjected to secondary extraction with DCM (1.5 mL/min, 750 mL) followed by phase separation in a tenth online gravity separation column (t_{R8-4}=3 min) to collect a tenth lower phase (with a flow rate controlled by a peristaltic pump: V=1.5 mL/min). The ninth lower phase and the tenth lower phase were conveyed to a fifth concentration device for 8-fold concentration (with parameters set as follows: V=0.5 mL/min, temperature T=75° C.; nitrogen flow rate F=70 mL/min), so as to obtain a mixed DCM/DMF solution containing a compound 6 as a feed liquid O. A total residence time was 43.5 min.

Step (h) m-CPBA (94.9 mM) was dissolved in DCM/DMF (0.5 mL/min, 250 mL) to obtain a feed liquid P. The feed liquid O (0.5 mL/min) and the feed liquid P were pumped into an eighteenth micromixer by a sixth plunger pump for mixing to obtain an eighth mixture. The eighth mixture was conveyed to an eighth microreactor (V₉=10 mL) for C6, C7 epoxidation reaction at 90° C. under a back pressure of 4.0 bar with a residence time of 10 min, so as to obtain an eighth reaction solution. The eighth reaction solution was quenched with an aqueous Na₂S₂O₃ solution (1.0 mL/min, 500 mL) in a nineteenth micromixer to remove benzoic acid, and further quenched with a 1 wt. % aqueous NaOH solution (1.0 mL/min, 500 mL) in a twentieth micromixer to remove residual m-CPBA, followed by extraction (t_{R9-1}=1 min) and phase separation in an eleventh online gravity separation column (t_{R9-2}=10 min) to obtain an eleventh lower phase (with a flow rate controlled by a peristaltic pump: V=4.0 mL/min) and a third upper phase. The third upper phase was subjected to secondary extraction with DCM (2.0 mL/min, 1000 mL) followed by phase separation in a twelfth online gravity separation column (t_{R9-4}=2 min) to collect a twelfth lower phase (with a flow rate controlled by a peristaltic pump: V=2.0 mL/min). The eleventh lower phase and the twelfth lower phase were conveyed to a sixth concentration device for 6-fold concentration (with parameters set as follows: V=1.0 mL/min, T=60° C.; nitrogen flow rate F=60 mL/min), so as to obtain a DCM solution containing a compound 14 as a feed liquid Q. A total residence time was 25 min.

Step (i) DMA·HCl (177.13 mM) was dissolved in DMF (0.5 mL/min, 1000 mL) to obtain a feed liquid R. The feed liquid Q and the feed liquid R were pumped into a twenty-first micromixer by a seventh plunger pump for mixing to obtain a ninth mixture. The ninth mixture was conveyed to a ninth microreactor for C6,C7 epoxide ring-opening reaction, C6 chlorination, dehydration and deprotection cascade reaction at 170° C. under a back pressure of 8.0 bar with a residence time of 40 min, so as to obtain a ninth reaction solution. The ninth reaction solution was quenched with a 1 wt. % aqueous NaOH solution (1.0 mL/min, 500 mL) in a twenty-second micromixer, then extracted with DCM (2.0 mL/min, 1000 mL), and subjected to phase separation in a thirteenth online gravity separation column (t_{R10-2}=10 min) to obtain a thirteenth lower phase (with a flow rate controlled by a peristaltic pump: V=3.0 mL/min) and a fourth upper phase. The fourth upper phase was subjected to secondary extraction with DCM (2.0 mL/min, 1000 mL) followed by phase separation in a fourteenth online gravity separation column (t_{R10-4}=3 min) to collect a fourteenth lower phase (with a flow rate controlled by a peristaltic pump: V=2.0 mL/min). The thirteenth lower phase and the fourteenth lower phase were mixed with an aqueous NaCl solution in a twenty-third micromixer, and then subjected to phase separation in a fifteenth online gravity separation column (t_{R10-6}=10 min) to collect a fifteenth lower phase (with a flow rate controlled by a peristaltic pump: V=5.0 mL/min). The fifteenth lower phase was conveyed to a seventh concentration device for 10-fold concentration (with parameters set as follows: V=0.5 mL/min, T=100° C.; nitrogen flow rate F=90 mL/min), and then loaded onto a fourth stainless steel column filled with Na₂SO₄ and SiO₂ in a weight ratio of 1:1 for moisture and impurity removal, so as to obtain a DCM solution containing a compound 5 as a feed liquid S. A total residence time was 107 min.

Step (j) Isopropenyl acetate (750 mL, 1.5 mL/min) and the feed liquid S (0.5 mL/min) were pumped into a twenty-fourth micromixer by an eighth plunger pump for mixing to obtain a tenth mixture. The tenth mixture was conveyed to a tenth microreactor (V₁₁=30 mL) containing 2.5 g of Amberlyst 15 acidic resin for acetylation reaction at 40° C. with a residence time of 25 min, so as to obtain a tenth reaction solution. The tenth reaction solution was quenched with a saturated aqueous NaHCO₃ solution (1.0 mL/min, 500 mL) in a twenty-fifth micromixer, then subjected to phase separation in a sixteenth online gravity separation column (t_{R11-2}=10 min) to collect a fifth upper phase (with a flow rate controlled by a peristaltic pump: V-2.0 mL/min). The fifth upper phase was a mixed DCM/isopropenyl acetate solution containing a compound 4. A total residence time was 37 min.

Cyproterone acetate 4 is a white powder, whose melting point is 206.5° C.-207.5° C. 1H NMR (400 MHZ, CDCl₃) δ 6.20 (d, J=2.3 Hz, 1H), 6.15 (s, J=1.0 Hz, 1H), 2.98 (ddd, J=15.7, 10.9, 1.8 Hz, 1H), 2.37-2.25 (m, 1H), 2.05 (s, 3H), 2.04-1.94 (m, 3H), 1.93 (d, J=2.9 Hz, 1H), 1.91-1.86 (m, 1H), 1.86-1.82 (m, 1H), 1.83-1.76 (m, 1H), 1.71 (td, J=7.8, 6.4 Hz, 1H), 1.66-1.59 (m, 1H), 1.55 (dd, J=12.6, 4.0 Hz, 1H), 1.52-1.37 (m, 2H), 1.27 (td, J=9.0, 7.9, 4.9 Hz, 1H), 1.21 (s, 3H), 0.87 (dd, J=6.4, 4.5 Hz, 1H), 0.71 (s, 3H). ¹³C NMR (101 MHZ, CDCl₃) δ 203.8, 198.1, 170.6, 152.3, 136.6, 130.2, 120.4, 96.2, 48.7, 47.7, 47.2, 38.7, 38.3, 31.0, 30.3, 26.4, 26.1, 25.3, 23.2, 22.9, 21.2, 20.8, 14.2, 12.4. Fourier transform infrared spectroscopy (FT-IR): nₘₐₓ=1740, 1718, 1651, 1370, 1351, 1257, 1245, 962, 898, 584 cm⁻¹. High-resolution mass spectrometry (HRMS, electrospray ionization mode) shows a mass-to-charge ratio (m/z) of 417.1832, which is consistent with the theoretical molecular formula C₂₄H₃₀ClO₄ of cyproterone acetate and its corresponding theoretical molecular weight of 417.1827. The specific rotations of the product measured at 25° C. and 20° C. are [α]_{D}²⁵=144.75 (c=0.13, chloroform) and [α]_{D}²⁰=156.45 (c=1.0, acetone), respectively.

By reasonably combining micromixers, microreactors, and online post-reaction treatment devices according to the synthetic process characteristics of cyproterone acetate, the present disclosure achieves efficient and rapid synthesis of cyproterone acetate. Continuous synthesis of cyproterone acetate is realized through a self-designed solvent switching device, which significantly improves production efficiency. The synthetic device eliminates the need for isolation and purification of synthetic intermediates, effectively reduces the generation of three wastes (industrial wastewater, waste gas, and solid waste), lowers production costs, and causes little environmental pollution. Based on the innovative concept of the present disclosure, any other changes and modifications made by those skilled in the art without departing from the spirit of the application shall fall within the scope of this application defined by the appended claims.

What is claimed is:

1. A fully-continuous synthesis method of cyproterone acetate, comprising:
(a) dissolving 4-androstene-3,17-dione and an electron acceptor in a first organic solvent to obtain a first feed liquid; and dispersing a first catalyst in a buffer solution to obtain a second feed liquid, wherein the first catalyst is an enzyme; and
pumping, by a first plunger pump, the first feed liquid and the second feed liquid into a first micromixer for mixing to obtain a first mixture, and conveying the first mixture to a dynamic tubular reactor and a first microreactor for 41-dehydrogenation reaction to obtain a first reaction solution; subjecting the first reaction solution to extraction with an extraction solvent in an extraction device followed by phase separation in a first online gravity separation column and upper phase removal to collect a first lower phase; concentrating the first lower phase in a first concentration device to obtain a first concentrate; and loading the first concentrate onto a first stainless steel column filled with Na₂SO₄ and SiO₂ in a weight ratio of 1:1 for water and impurity removal to obtain an androstene-1,4-dien-3,17-dione (10)-containing mixed solution as a third feed liquid;
(b) dissolving ethynylmagnesium bromide in a second organic solvent to obtain a fourth feed liquid; pumping, by a second plunger pump, the third feed liquid and the fourth feed liquid into a second micromixer for mixing to obtain a second mixture; conveying the second mixture to a second microreactor for alkynylation reaction to obtain a second reaction solution; and quenching the second reaction solution with an aqueous HCl solution in a third micromixer followed by extraction and phase separation in a second online gravity separation column to collect a second lower phase as a fifth feed liquid, wherein the second lower phase is a Δ^{1,4}-dien-3-one propargyl alcohol (12)-containing mixed solution;
(c) dissolving an acidic reagent in dichloroethane to obtain a sixth feed liquid; pumping, by a third plunger pump, the fifth feed liquid and the sixth feed liquid into a fourth micromixer for mixing to obtain a third mixture; conveying the third mixture to a third microreactor for Rupe rearrangement to obtain a third reaction solution; and conveying the third reaction solution to a fifth micromixer through a first back pressure regulator, quenching the third reaction solution with a first aqueous NaHCO₃ solution in the fifth micromixer followed by extraction and phase separation in a third online gravity separation column to collect a third lower phase as a seventh feed liquid, wherein the third lower phase is a Δ^{1, 4, 16}-trien-3,20-dione (9)-containing mixed solution;
(d) dissolving a second catalyst and PhSiH₃ in a third organic solvent to obtain an eighth feed liquid; pumping, by a fourth plunger pump, the seventh feed liquid, the eighth feed liquid and oxygen into a sixth micromixer for mixing to obtain a fourth mixture, wherein the oxygen is stored in an oxygen cylinder with flow rate regulated by a check valve and a gas flow meter; conveying the fourth mixture to a fourth microreactor for Mukaiyama hydration reaction followed by mixing with an ethanol solution of P(OEt)₃ in a seventh micromixer to complete the Mukaiyama hydration reaction, so as to obtain a fourth reaction solution; quenching the fourth reaction solution with a first aqueous sodium chloride solution in an eighth micromixer followed by extraction and phase separation in a fourth online gravity separation column to collect a fourth lower phase; and mixing the fourth lower phase with a first N,N-dimethylformamide (DMF) solution in a ninth micromixer followed by concentration in a second concentration device to obtain a 17α-hydroxy-Δ^{1,4}-dien-3,20-dione (8)-containing mixed solution as a ninth feed liquid;
(e) dissolving tetrachloro-p-benzoquinone (TCBQ) in a fourth organic solvent to obtain a tenth feed liquid; pumping, by a fifth plunger pump, the ninth feed liquid and the tenth feed liquid into a tenth micromixer for mixing to obtain a fifth mixture; conveying the fifth mixture to a fifth microreactor for 46-dehydrogenation reaction to obtain a fifth reaction solution; quenching the fifth reaction solution with a first 1 wt. % aqueous NaOH solution in an eleventh micromixer followed by extraction with DCM and phase separation in a fifth online gravity separation column to obtain a fifth lower phase and a first upper phase; subjecting the first upper phase to secondary extraction with DCM followed by phase separation in a sixth online gravity separation column to collect a sixth lower phase; mixing the fifth lower phase and the sixth lower phase with a second aqueous sodium chloride solution in a twelfth micromixer followed by phase separation in a seventh online gravity separation column to collect a seventh lower phase; and concentrating the seventh lower phase in a third concentration device followed by loading onto a second stainless steel column filled with Na₂SO₄ and SiO₂ in a weight ratio of 1:1 for moisture and impurity removal, so as to obtain a 17α-hydroxy-Δ^{1,4,6}-trien-3,20-dione (13)-containing dichloromethane solution as an eleventh feed liquid;
(f) dissolving a third catalyst, ethylene glycol and a water absorbent in dichloromethane to obtain a twelfth feed liquid; mixing the eleventh feed liquid with the twelfth feed liquid in a thirteenth micromixer to obtain a sixth mixture; conveying the sixth mixture to a sixth microreactor for ketal protection reaction to obtain a sixth reaction solution; conveying the sixth reaction solution to a fourteenth micromixer through a second back pressure regulator, and quenching the sixth reaction solution with a second aqueous NaHCO₃ solution in the fourteenth micromixer followed by phase separation in an eighth online gravity separation column to collect an eighth lower phase; mixing the eighth lower phase with a second DMF solution in a fifteenth micromixer followed by concentration in a fourth concentration device to obtain a second concentrate; and loading the second concentrate onto a third stainless steel column filled with Na₂SO₄ and SiO₂ in a weight ratio of 1:1 for moisture and impurity removal, so as to obtain a 17α-hydroxy-Δ^{1,4,6}-trien-3-one-20-ketal (7)-containing mixed solution as a thirteenth feed liquid;
(g) under a nitrogen atmosphere, dissolving trimethylsulfoxonium iodide (TMSOI) and a base in DMF followed by reaction at room temperature for 0.6-1.2 h to obtain a fourteenth feed liquid; mixing the thirteenth feed liquid with the fourteenth feed liquid in a sixteenth micromixer to obtain a seventh mixture; conveying the seventh mixture to a seventh microreactor for C1,C2 cyclopropanation reaction to obtain a seventh reaction solution; quenching the seventh reaction solution with a third aqueous sodium chloride solution in a seventeenth micromixer followed by extraction with dichloromethane and phase separation in a ninth online gravity separation column to obtain a ninth lower phase and a second upper phase; subjecting the second upper phase to secondary extraction with dichloromethane followed by phase separation in a tenth online gravity separation column to collect a tenth lower phase; and conveying the ninth lower phase and the tenth lower phase to a fifth concentration device for concentration, so as to obtain a 17α-hydroxy-1α,2α-cyclopropa-Δ^{4,6}-dien-3-one-20-ketal (6)-containing mixed solution as a fifteenth feed liquid;
(h) dissolving an oxidant in dichloromethane to obtain a sixteenth feed liquid; pumping, by a sixth plunger pump, the fifteenth feed liquid and the sixteenth feed liquid into an eighteenth micromixer for mixing to obtain an eighth mixture; conveying the eighth mixture to an eighth microreactor for C6, C7 epoxidation reaction to obtain an eighth reaction solution; conveying the eighth reaction solution to a nineteenth micromixer through a third back pressure regulator, and quenching the eighth reaction solution sequentially with an aqueous Na₂S₂O₃ solution in the nineteenth micromixer and a second 1 wt. % aqueous NaOH solution in a twentieth micromixer to remove residual oxidant followed by phase separation in an eleventh online gravity separation column to obtain an eleventh lower phase and a third upper phase; subjecting the third upper phase to secondary extraction with dichloromethane followed by phase separation in a twelfth online gravity separation column to collect a twelfth lower phase; and conveying the eleventh lower phase and the twelfth lower phase to a sixth concentration device for concentration, so as to obtain a 17α-hydroxy-6α,7α-epoxy-1α,2α-cyclopropa-Δ⁴-en-3-one-20-ketal (14)-containing mixed solution as a seventeenth feed liquid;
(i) dissolving N,N-dimethylacetamide hydrochloride (DMA·HCl) in DMF to obtain an eighteenth feed liquid; pumping, by a seventh plunger pump, the seventeenth feed liquid and the eighteenth feed liquid into a twenty-first micromixer for mixing to obtain a ninth mixture; conveying the ninth mixture to a ninth microreactor for C6, C7 epoxide ring-opening reaction, C6 chlorination, dehydration and deprotection cascade reaction to obtain a ninth reaction solution; conveying the ninth reaction solution to a twenty-second micromixer through a fourth back pressure regulator, and quenching the ninth reaction solution with a third 1 wt. % aqueous NaOH solution in the twenty-second micromixer followed by extraction with dichloromethane and phase separation in a thirteenth online gravity separation column to obtain a thirteenth lower phase and a fourth upper phase; subjecting the fourth upper phase to secondary extraction with dichloromethane followed by phase separation in a fourteenth online gravity separation column to collect a fourteenth lower phase; mixing the thirteenth lower phase and the fourteenth lower phase with a fourth aqueous sodium chloride solution in a twenty-third micromixer followed by phase separation in a fifteenth online gravity separation column to collect a fifteenth lower phase; and concentrating the fifteenth lower phase in a seventh concentration device followed by loading onto a fourth stainless steel column filled with Na₂SO₄ and SiO₂ in a weight ratio of 1:1 for moisture and impurity removal, so as to obtain a 17α-hydroxy-1α,2α-cyclopropa-6-chloro-Δ^{4,6}-dien-3,20-dione (5)-containing mixed solution as a nineteenth feed liquid; and
(j) pumping, by an eighth plunger pump, the nineteenth feed liquid and an acetylation reagent into a twenty-fourth micromixer for mixing to obtain a tenth mixture; conveying the tenth mixture to a tenth microreactor containing an acid catalyst for acetylation reaction to obtain a tenth reaction solution; quenching the tenth reaction solution with a third aqueous NaHCO₃ solution in a twenty-fifth micromixer followed by phase separation in a sixteenth online gravity separation column to collect a fifth upper phase, wherein the fifth upper phase is a cyproterone acetate (4)-containing mixed solution; and
subjecting the fifth upper phase to chromatographic separation to obtain a pure cyproterone acetate product.

2. The fully-continuous synthesis method of claim 1, wherein in step (a):
a flow rate ratio of 4-androstene-3,17-dione to the electron acceptor to the first catalyst in the dynamic tubular reactor is controlled such that a molar ratio of the electron acceptor to 4-androstene-3,17-dione to the first catalyst is 0.1-0.3:1:1.0-5.0;
the electron acceptor is selected from the group consisting of phenazine methosulfate, 2,6-dichlorophenolindophenol, resazurin, N,N,N′,N′-tetramethyl-p-phenylenediamine, tetramethylthionine chloride, coenzyme Q, vitamin K and menadione;
the enzyme is a 3-ketosteroid-Δ¹-dehydrogenase (Δ¹-KstD) or a mutant thereof;
the first organic solvent is selected from the group consisting of dichloromethane, 1,2-dichloroethane, methanol, N,N-dimethylacetamide, dimethyl sulfoxide, tetrahydrofuran (THF) and 1,4-dioxane;
the buffer solution is selected from the group consisting of a phosphate buffer, a glycine-sodium hydroxide buffer, a Tris-hydrochloric acid buffer, a phthalic acid-hydrochloric acid buffer and a glycine-hydrochloric acid buffer;
the extraction solvent is dichloromethane or dichloroethane;
a temperature of the dynamic tubular reactor and a temperature of the first microreactor are each controlled at 20° C.-45° C.; a residence time of the first mixture in the dynamic tubular reactor is 30-120 min; and a residence time of the first mixture in the first microreactor is 22-90 min;
the extraction of the first reaction solution is performed for 1-5 min; and
the phase separation in the first online gravity separation column is performed for 3-20 min; and a residence time of the first lower phase in the first concentration device is 1-10 min.

3. The fully-continuous synthesis method of claim 2, wherein in step (b):
a molar concentration of ethynylmagnesium bromide is 100-300 mmol/L;
the second organic solvent is one or two selected from the group consisting of THE, N,N-dimethylacetamide (DMAC), dimethyl sulfoxide (DMSO) and hexamethylphosphoramide (HMPA); and
a temperature of the second microreactor is controlled at 20° C.-45° C.; and a residence time of the second mixture in the second microreactor is 30-120 min;
in step (c):
a molar concentration of the acidic reagent is 50-200 mmol/L;
the acidic reagent is selected from the group consisting of concentrated sulfuric acid, trifluoromethanesulfonic acid, an Eaton's reagent, methanesulfonic acid, an Amberlyst 15 acidic resin, p-toluenesulfonic acid, aluminum trichloride and acetic acid; and
a temperature of the third microreactor is controlled at 60° C.-100° C.; a molar ratio of the acidic reagent to Δ^{1, 4, 16}-trien-3,20-dione in the third lower phase is 1.5-3:1; and a residence time of the third mixture in the third microreactor is 20-30 min.

4. The fully-continuous synthesis method of claim 3, wherein in step (d):
a molar concentration of the second catalyst is 1-3 mol %;
the second catalyst is selected from the group consisting of Mn(dpm)₃, Co(acac)₂, Fe(dpm)₃, Fe(acac)₃, Fe(acac)₂, Co(acac)₃, cobalt acetate, cobalt (II) bromide, cobalt chloride and cobalt iso-octoate;
the third organic solvent is one or two selected from the group consisting of dichloromethane, 1,2-dichloroethane, methanol, ethanol and THF; and
a temperature of the fourth microreactor is controlled at 25° C.-35° C.; and a residence time of the fourth mixture in the fourth microreactor is 10-15 min;
in step (e):
the fourth organic solvent is selected from the group consisting of dichloromethane, 1,2-dichloroethane, DMAC, DMSO, 1,4-dioxane and tert-butanol; and
a temperature of the fifth microreactor is controlled at 100° C.-140° C.; and a residence time of the fifth mixture in the fifth microreactor is 4-15 min;
in step (f):
the water absorbent is selected from the group consisting of a silica gel, a molecular sieve, anhydrous sodium sulfate, anhydrous magnesium sulfate, anhydrous calcium chloride and trimethyl orthoformate (TMOF);
the third catalyst is selected from the group consisting of concentrated sulfuric acid, p-toluenesulfonic acid, BF₃·Et₂O, TiCl₄, Bi(NO₃)₃, LiBF₄, Cu(BF₄)₂, RuCl₃·H₂O, CoCl₂, InCl₃, In(OTf)₃, ZrCl₄ and Zn(BF₄)₂; and
a temperature of the sixth microreactor is controlled at 40° C.-100° C.; and a residence time of the sixth mixture in the sixth microreactor is 15-20 min.

5. The fully-continuous synthesis method of claim 4, wherein in step (g):
the base is selected from the group consisting of lithium bis(trimethylsilyl)amide (LiHMDS), 1,2-dianilinoethane (NODX), potassium tert-butoxide (′BuOK), lithium diisopropylamide (LDA), sodium methoxide (MeONa) and NaH;
a temperature of the seventh microreactor is controlled at 25° C.-40° C.; and a residence time of the seventh mixture in the seventh microreactor is 15-20 min; and
a molar ratio of TMSOI to 17α-hydroxy-1α,2α-cyclopropa-Δ^{4,6}-dien-3-one-20-ketal in the fifteenth feed liquid is 2-3:1, and a molar ratio of the base to 17α-hydroxy-1α,2α-cyclopropa-Δ^{4,6}-dien-3-one-20-ketal in the fifteenth feed liquid is 1.5-3.0:1;
in step (h):
the oxidant is selected from the group consisting of performic acid, peracetic acid, perbenzoic acid, m-CPBA, trifluoroperacetic acid and hydrogen peroxide; and
a temperature of the eighth microreactor is controlled at 25° C.-120° C.; and a residence time of the eighth mixture in the eighth microreactor is 10-20 min;
in step (i):
the steroidal C6, C7 epoxide ring-opening reaction, the C6 chlorination, the dehydration and the deprotection cascade reaction are performed as a one-pot reaction in the same reaction system; and
a temperature of the ninth microreactor is controlled at 80° C.-180° C.; and a residence time of the ninth mixture in the ninth microreactor is 20-40 min;
in step (j):
the acetylation reagent is selected from the group consisting of acetic acid, acetic anhydride, acetyl chloride, trifluoroacetic anhydride and isopropenyl acetate; and
the acid catalyst is selected from the group consisting of concentrated sulfuric acid, trifluoromethanesulfonic acid, methanesulfonic acid, an Amberlyst 15 acidic resin, p-toluenesulfonic acid and acetic acid.

6. The fully-continuous synthesis method of claim 1, wherein each of the first micromixer, the second micromixer, the third micromixer, the fourth micromixer, the fifth micromixer, the sixth micromixer, the seventh micromixer, the eighth micromixer, the ninth micromixer, the tenth micromixer, the eleventh micromixer, the twelfth micromixer, the thirteenth micromixer, the fourteenth micromixer, the fifteenth micromixer, the sixteenth micromixer, the seventeenth micromixer, the eighteenth micromixer, the nineteenth micromixer, the twentieth micromixer, the twenty-first micromixer, the twenty-second micromixer, the twenty-third micromixer, the twenty-fourth micromixer and the twenty-fifth micromixer is one of a T-type micromixer, a Y-type micromixer, a cross-type mixer, a coaxial flow micromixer and a flow-focusing micromixer;
each of the first microreactor, the second microreactor, the third microreactor, the fourth microreactor, the fifth microreactor, the sixth microreactor, the seventh microreactor, the eighth microreactor, the ninth microreactor and the tenth microreactor is a tubular microchannel reactor or a plate-type microchannel reactor;
each of the first concentration device, the second concentration device, the third concentration device, the fourth concentration device, the fifth concentration device, the sixth concentration device and the seventh concentration device is a tube-in-tube concentration device; and
each of the first online gravity separation column, the second online gravity separation column, the third online gravity separation column, the fourth online gravity separation column, the fifth online gravity separation column, the sixth online gravity separation column, the seventh online gravity separation column, the eighth online gravity separation column, the ninth online gravity separation column, the tenth online gravity separation column, the eleventh online gravity separation column, the twelfth online gravity separation column, the thirteenth online gravity separation column, the fourteenth online gravity separation column, the fifteenth online gravity separation column and the sixteenth online gravity separation column adopts a glass tube gravity liquid-liquid separation device.

7. The fully-continuous synthesis method of claim 1, each of the first concentration device, the second concentration device, the third concentration device, the fourth concentration device, the fifth concentration device, the sixth concentration device and the seventh concentration device is composed of two feed liquid containers, a solvent storage tank, a recovery tank, a nitrogen cylinder, a concentration tube, a peristaltic pump, a hose, a T-type connector and a mass flow controller connected in series; the two feed liquid containers are connected via the T-type connector; the peristaltic pump is configured to pump a corresponding feed liquid into a polytetrafluoroethylene coil for mixing to obtain a corresponding mixture, so that the corresponding mixture enters the concentration tube along with N₂ via the T-type connector for heating and purging concentration to obtain a volatile solvent, the volatile solvent in the concentration tube enters a condenser to obtain a condensate, and the condensate enters the recovery tank to recover an evaporated solvent; and the peristaltic pump is configured such that after the corresponding feed liquid reaches a required concentration multiple, the corresponding feed liquid is transported by the peristaltic pump to a next operation stage.

8. The fully-continuous synthesis method of claim 1, the first back pressure regulator, the second back pressure regulator, the third back pressure regulator and the fourth back pressure regulator are each made of stainless steel or Hastelloy; and the first back pressure regulator, the second back pressure regulator, the third back pressure regulator and the fourth back pressure regulator each have a connecting pipeline size of 1.6-20 mm and a pressure range of 0.1-2.0 MPa.
